SEQUENCE LISTING (3 sequences)

SEQ ID NO: 1 - protein, 11 aa, synthetic construct
GPVSAVLTELR

SEQ ID NO: 2 - protein, 8 aa, synthetic construct
IIIEELPK

SEQ ID NO: 3 - protein, 15 aa, synthetic construct
GPLASQISGLYLPYK

(19) United States
(12) Patent Application Publication
(10) Pub. No.: US 2026/0199399 A1
(43) Pub. Date: Jul. 16, 2026

(54) CARDIAC CELL COMPOSITIONS, USES THEREOF, AND METHODS FOR TREATMENT OF HEART DISEASE

(71) Applicant: MaineHealth, Portland, ME (US)

(72) Inventors: Douglas B. Sawyer, Cape Elizabeth, ME (US); Sergey Ryzhov, Cape Elizabeth, ME (US)

(73) Assignee: MaineHealth, Portland, ME (US)

(21) Appl. No.: 19/448,586

(22) Filed: Jan. 14, 2026

Related U.S. Application Data
(60) Provisional application No. 63/745,482, filed on Jan. 15, 2025.

(51) Int. Cl. A61K 35/34; A61K 9/00; A61P 9/10; C12N 5/077

(52) U.S. Cl. CPC A61K 35/34; A61K 9/0019; A61P 9/10; C12N 5/0657; C12N 2500/84

(57) ABSTRACT

The present invention provides compositions and methods of use thereof in the treatment of heart disease. In some embodiments, the compositions comprise highly proliferative cells, which can express CXCL6, CTHRC1, and CD73, and can be improve cardiac function when administered to a subject.

### RELATED APPLICATIONS

The instant application claims priority to U.S. Provisional Application No. 63/745,482, filed Jan. 15, 2025, the entire contents of which are expressly incorporated by reference herein.

### GOVERNMENT SUPPORT

This invention was made with government support under grant number R01HL139887, awarded by the National Institutes of Health/National Heart, Lung, and Blood Institute. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 9, 2026, is named “136191-00602.xml” and is 3,531 bytes in size. The sequence listing contained in this .XML file is part of the specification and is hereby incorporated by reference herein in its entirety.

### FIELD OF DISCLOSURE

The present disclosure relates to treatments for a disease or disorder, such as, but not limited to heart disease.

### BACKGROUND OF THE INVENTION

The role of specific cell types in cardiac recovery from injury remains incompletely understood. Moreover, transplantation of specific adult cardiac cells has been investigated as a therapeutic approach to treat various cardiovascular diseases (CVD)¹. A population of highly proliferative cells (hHiPCs) derived from cells in the adult human heart hold promising therapeutic potential². To date, no study has investigated the role of this cell population in cardiac repair after myocardial infarction (MI) and its potential efficacy in cellular therapeutic approaches to improve cardiac repair.

### SUMMARY OF THE INVENTION

Accordingly, in one aspect, the present invention provides a composition of a plurality of highly proliferative cells, wherein the plurality of highly proliferative cells can express one or more of CXCL6, CTHRC1, and CD73, and wherein the highly proliferative cells are generated by clonal expansion of a cell.

In one embodiment, the cell can be a stem cell or a differentiated cell. In one embodiment, the stem cell is an induced pluripotent stem cell (iPSC). In one embodiment, the cell can be a heart cell.

In one embodiment, the heart cell can be an epicardial cell, a pericardial cell, a cardiomyocyte, a cardiac endothelial cell, a cardiac fibroblast cell, cardiac smooth muscle cells, cardiac immune cells and an endocardial cell.

In one embodiment, the cell can be derived from a biopsy. In one embodiment, the biopsy can be from a human heart. In one embodiment, the biopsy can be from human ventricle epicardial tissues.

In one embodiment, further the plurality of highly proliferative cells can express one or more of 1B46, CREG1, MRC1, TFPI2, CD200, PLXB2, or LRP1.

In one embodiment, further the plurality of highly proliferative cells can express one or more of ACTN4, APPL1, CAPZA1, CNN2, COL1A2, HIST2H3D, ISG15, MAOA, MTAP, MX1, PSMA2, PSMB4, PSMB7, PSMB9, PSME1, PYCARD, RIPK2, STAT1, STAT2, or YAP.

In one embodiment, further the plurality of highly proliferative cells can express one or more CXCL5, CXCL1, CXCL3, CXCL8, AGRIN, or CO3.

In one embodiment, further the plurality of highly proliferative cells do not express c-kit and/or CD34. In one embodiment, the plurality of highly proliferative cells do not comprise CD45+ cells.

In one embodiment, the highly proliferative cells can be between about 25 μm to about 110 μm in size across at least one axis. In one embodiment, the highly proliferative cells can be between about 50 μm to about 110 μm in size across at least one axis.

In one embodiment, a concentration of a culture of the highly proliferative cells can increase by at least 2-fold in about 15 hours to about 90 hours. In one embodiment, a concentration of a culture of the highly proliferative cells can increase by at least 2-fold in less than about 90 hours, less than about 80 hours, less than about 70 hours, less than about 60 hours, less than about 50 hours, less than about 40 hours, less than about 30 hours, less than about 20 hours, or less than about 15 hours.

In one embodiment, further the composition can include a medium suitable for maintaining the viability of the highly proliferative cells.

In one embodiment, the composition can be produced according to a method including: providing a cell; culturing the heart cell to form a colony; selecting a colony, wherein the cells of the colony are between about 50 μm to about 110 μm in size, and wherein a concentration of a culture of the cells of the colony increases by at least 2-fold in about 15 hours to about 90 hours.

In one embodiment, the cell can be a stem cell or a differentiated cell. In one embodiment, the stem cell can be an induced pluripotent stem cell (iPSC). In one embodiment, the cell can be a heart cell. In one embodiment, the heart cell can be an epicardial cell, a pericardial cell, a cardiomyocyte, a cardiac endothelial cell, a cardiac fibroblast cell, and an endocardial cell.

In one embodiment, the cell can be cultured in a medium comprising fetal bovine serum (FBS) and an endothelial cell growth media.

In one embodiment, the cell can be derived from a biopsy. In one embodiment, the biopsy can be from a human heart. In one embodiment, the biopsy can be from human ventricle epicardial tissues.

In one embodiment, CD45+ cells can be depleted from the composition.

In one embodiment, the composition can include between about 0.25×10⁵ cells to about 5.0×10⁵ cells. In one embodiment, the composition can include about 2.5×10⁵ cells.

In one embodiment, the cell can be obtained from a subject to be treated.

In another aspect, the present invention provides a pharmaceutical composition comprising any one of the compositions disclosed herein, and a pharmaceutically acceptable carrier.

In one embodiment, the pharmaceutically acceptable excipient can be suitable for intramyocardial injection.

In another aspect, the present invention provides a method of producing any one of the compositions disclosed herein, the method including: providing a cell; culturing the cell to form a colony; selecting a colony, wherein the cells of the colony are between about 50 μm to about 110 μm in size, and wherein a concentration of a culture of the cells of the colony increases by at least 2-fold in about 15 hours to about 90 hours; and depleting CD45+ cells from the composition.

In another aspect, the present invention provides a method of producing the composition comprising a plurality of highly proliferative cells, the method including: providing a cell; culturing the cell to form a colony; selecting a colony, wherein the cells of the colony are between about 50 μm to about 110 μm in size, and wherein a concentration of a culture of the cells of the colony increases by at least 2-fold in about 15 hours to about 90 hours; and depleting CD45+ cells from the composition.

In one embodiment, the cell can be a stem cell or a differentiated cell. In one embodiment, the stem cell can be an induced pluripotent stem cell (iPSC). In one embodiment, the cell can be a heart cell. In one embodiment, the heart cell can be an epicardial cell, a pericardial cell, a cardiomyocyte, a cardiac endothelial cell, a cardiac fibroblast cell, and an endocardial cell.

In one embodiment, the cell can be derived from a biopsy. In one embodiment, the biopsy can be from a human heart. In one embodiment, the biopsy can be from human ventricle epicardial tissues. In one embodiment, the cell can be obtained from a subject to be treated.

In one embodiment, further the plurality of highly proliferative cells can express one or more of 1B46, CREG1, MRC1, TFPI2, CD200, PLXB2, or LRP1. In one embodiment, further the plurality of highly proliferative cells can express one or more of ACTN4, APPL1, CAPZA1, CNN2, COL1A2, HIST2H3D, ISG15, MAOA, MTAP, MX1, PSMA2, PSMB4, PSMB7, PSMB9, PSME1, PYCARD, RIPK2, STAT1, STAT2, or YAP. In one embodiment, further the plurality of highly proliferative cells can express one or more CXCL5, CXCL1, CXCL3, CXCL8, AGRIN, or CO3.

In one embodiment, further the plurality of highly proliferative cells do not express c-kit and/or CD34.

In one embodiment, the cell can be cultured in a medium comprising fetal bovine serum (FBS) and an endothelial cell growth media.

In another aspect, the present invention provides a method of treating heart disease in a subject, the method comprising administering to the subject any one of the compositions disclosed herein, the any one of the pharmaceutical compositions disclosed herein, or any one of the compositions produced by any one of the methods disclosed herein.

In another aspect, the present invention provides a method of improving cardiac function after myocardial infarction in a subject, the method comprising administering to the subject any one of the compositions disclosed herein, the any one of the pharmaceutical compositions disclosed herein, or any one of the compositions produced by any one of the methods disclosed herein.

In one embodiment, cardiac function can be measured by fractional shortening. In one embodiment, fractional shortening can be increased by at least 5%, at least 10%, at least 15%, at least 20%, at least 25%, at least 30%, at least 35%, at least 40%, or at least 45% compared to fractional shortening prior to administration of the composition. In one embodiment, fractional shortening can be increased between about 5% and about 50%, about 5% and about 45%, about 5% and about 40%, about 5% and about 35%, about 5% and about 30%, about 5% and about 25%, about 5% and about 20%, about 5% and about 15%, about 5% and about 10%, about 10% and about 50%, about 10% and about 45%, about 10% and about 40%, about 10% and about 35%, about 10% and about 30%, about 10% and about 25%, about 10% and about 20%, about 10% and about 15%, about 15% and about 50%, about 15% and about 45%, about 15% and about 40%, about 15% and about 35%, about 15% and about 30%, about 15% and about 25%, about 15% and about 20%, about 20% and about 50%, about 20% and about 45%, about 20% and about 40%, about 20% and about 35%, about 20% and about 30%, about 20% and about 25%, about 25% and about 50%, about 25% and about 45%, about 25% and about 40%, about 25% and about 35%, about 25% and about 30%, about 30% and about 50%, about 30% and about 45%, about 30% and about 40%, about 30% and about 35%, about 35% and about 50%, about 35% and about 45%, about 35% and about 40%, about 40% and about 50%, about 40% and about 45%, or about 45% and about 50% compared to fractional shortening prior to administration of the composition.

In another aspect, the present invention provides a method of improving cardiac remodeling after myocardial infarction in a subject, the method comprising administering to the subject any one of the compositions disclosed herein, the any one of the pharmaceutical compositions disclosed herein, or any one of the compositions produced by any one of the methods disclosed herein.

In one embodiment, the composition can be administered to the subject at least 10 minutes, at least 30 minutes, at least 1 hour, at least 2 hours, at least 3 hours, at least 5 hours, at least 12 hours, at least 24 hours, at least 48 hours, at least 72 hours after a myocardial infarction.

In one embodiment, about 1% to about 5% of the total number of cells in the composition can be in the heart of the subject about 24 hours after administration.

In one embodiment, the composition can be injected into and/or adjacent to the subject's heart. In one embodiment, the composition can be injected into a peri-infarct area.

In one embodiment, at least 24 hours after administration of the composition CD45+ cells can increase at the site of administration by at least 1.5 fold, at least 2 fold, at least 2.5 fold, or at least 3 fold compared to the level of CD45+ cells at the site of administration prior to administration. In one embodiment, at least 24 hours after administration of the composition CD45+ cells can increase at the site of administration between about 1.5 fold to about 4 fold, about 1.5 fold to about 3.5 fold, about 1.5 fold to about 3 fold, about 1.5 fold to about 2.5 fold, about 1.5 fold to about 2 fold, about 2 fold to about 4 fold, about 2 fold to about 3.5 fold, about 2 fold to about 3 fold, about 2 fold to about 2.5 fold, about 2.5 fold to about 4 fold, about 2.5 fold to about 3.5 fold, about 2.5 fold to about 3 fold, about 3 fold to about 4 fold, about 3 fold to about 3.5 fold, or about 3.5 fold to about 4 fold compared to the level of CD45+ cells at the site of administration prior to administration.

In one embodiment, at least 24 hours after administration of the composition granulocytic phagocytes can increase at the site of administration by at least 1.5 fold, at least 2 fold, at least 2.5 fold, or at least 3 fold compared to the level of granulocytic phagocytes at the site of administration prior to administration. In one embodiment, at least 24 hours a after administration of the composition granulocytic phagocytes can increase at the site of administration between about 1.5 fold to about 4 fold, about 1.5 fold to about 3.5 fold, about 1.5 fold to about 3 fold, about 1.5 fold to about 2.5 fold, about 1.5 fold to about 2 fold, about 2 fold to about 4 fold, about 2 fold to about 3.5 fold, about 2 fold to about 3 fold, about 2 fold to about 2.5 fold, about 2.5 fold to about 4 fold, about 2.5 fold to about 3.5 fold, about 2.5 fold to about 3 fold, about 3 fold to about 4 fold, about 3 fold to about 3.5 fold, or about 3.5 fold to about 4 fold compared to the level of granulocytic phagocytes at the site of administration prior to administration.

In one embodiment, the granulocytic phagocytes can be CD11b+Ly6G+ cells.

### BRIEF DESCRIPTION OF THE FIGURES

FIGS. 1A-1E. LC-MS/MS characterization of hHiPCs compared to terminally differentiated endothelial cells displays pro-reparative signaling traits. hHiPCs (n=9) or HRECs (n=4) lysates were collected and proteins were quantified using LC-MS/MS with SWATH acquisition analysis. FIG. 1A. Proteomic analysis of proteins with differential regulation of proteins between hHiPC and ECs. Volcano plot illustrates upregulated (229, red; FC>2.0) and downregulated proteins (153, blue; FC<0.6). FIG. 1B. Tissue specificity and subcellular localization for detected proteins were determined using UniProt. FIG. 1C. GOrilla enrichment analysis of upregulated proteins in hHiPC were analyzed with GOrilla enrichment platform using FDR analysis (p<0.01). FIG. 1D. Heat map of cytokine-mediated signaling proteins upregulated in hHiPC (p<0.05, log FC>0.3). FIG. 1E. Ontologic analysis of top differentially expressed proteins with indicated signal peptides (UniProt) in hHiPC cell lysates and fold change (FC) comparisons compared to EC. All data were normalized using most likely ratio MLR normalization and p-value were determined by Welch's t-test.

FIGS. 2A-2D. Spectral identification evidence for key hHiPC cardiac repair paracrine factors. hHiPC (n=6) secretome samples were collected after 24-hours of reduced serum conditions (1%) and peptides were analyzed using LC-MS/MS analysis. FIG. 2A. Percent signal peptides detected compared to proteins with non-signal peptides. FIGS. 2B-2D. Spectra of highest confidence peptide (>99%) for proteins (FIG. 2B) CXCL6 (GPVSAVLTELR; SEQ ID NO: 1), (FIG. 2C) CTHRC1 (IIIEELPK; SEQ ID NO: 2), and (FIG. 2D) CD73 (GPLASQISGLYLPYK; SEQ ID NO: 3). Chromatogram of 6 characteristic ion liquid chromatographic elution profiles for the indicated peptide (top pane). Spectra of high-resolution m/z (blue lines) compared to theoretical m/z (pink lines) for individual m/z values for the given peptide (bottom pane). Detected y- and b-ions of peptide sequence are highlighted in the residue table. The cut-off for spectra analysis was 400-1250 m/z.

FIGS. 3A-311. Single Cell RNA-seq analysis of hHiPC with adult and neonatal CDC. On-line single-cell RNA-seq analysis of adult and neonatal CDC compared to highly expressed hHiPC protein markers (identified by the secretome analysis) identifies key genes involved in pro-reparative phenotype (CDC and hHiPC). FIG. 3A. UMAP of the indicated cell populations after filtering low abundance transcripts. FIG. 3B. Gene expression of CXCL6, CTHRC1, and NT5E (CD73) across indicated clusters. FIG. 3C. Gene expression analysis of transcripts corresponding to proteins identified by LC-MS/MS in hHiPC, compared across cardiac EC, CF, SMC, adult CDC, and neonatal CDC. Analyzed by Wilcoxon rank sum test with Benjamini-Hochberg correction for multiple testing (*** p<0.001, ns, not significant). FIG. 3D. Dot plot of average expression and percent cells expressed in all cardiac cell types. FIGS. 3E-311 hHiPC markers are not expressed in non-proliferating cardiac cells. GEO (GSE109816, GSE149699) single-cell RNA-seq analysis of adult and neonatal proliferating cells (CF, EC, SMC, AdultCDC, NeonatalCDC) and non-proliferating cardiomyocytes (CM) compared to highly expressed hHiPC protein markers (identified by the proteomic analysis) identifies key genes involved in pro-proliferative phenotype (CDC and hHiPC). FIG. 3E. UMAP of the indicated cell populations after normalization. FIG. 3F. Gene expression analysis of transcripts corresponding to proteins identified by LC-MS/MS in hHiPC and known CM specific markers (MYH6, MYH7, TNNT2). Adult and neonatal CDC compared to non-proliferating CMs was analyzed by Wilcoxon rank sum test with Benjamini-Hochberg correction for multiple testing (*** p<0.001). FIG. 3G. Gene expression of CXCL6, CTHRC1, and CD73 across indicated cell clusters. FIG. 3H. Dot plot of average gene expression and percent cells expressed in CM, adult CDC, and neonatal CDC. Proliferation markers were determined by go-term analysis (GO:0008284; positive regulation of cell proliferation) from hHiPC proteomic dataset.

FIGS. 4A-4K Transplanted hHiPC survive the acute inflammatory phase after MI. Cardiac cell suspensions were prepared with enzymatic digestion (collagenase I dispase II). FIG. 4A. Flow cytometric plots showing the overall approach taken to analyze all events (left), with the exclusion of dead cells and cell debris (SSC viability dye plot) and cell aggregates (FSC-H/A plot), followed by the examination of non-immune cells (right plot). FIG. 4B. Flow cytometric plots showing the efficiency of transplantation with injections of different numbers of human HiPC cells, including 0.25×10⁵, 0.50×10⁵, 1.0×10⁵, 2.5×10⁵, and 5.0×10⁵ per mouse heart (in 125 μL of PBS). Human cells were identified 24 hours after the cell injections using antibodies against human CD29/β1 integrin (cell surface) and human mitochondria (clone 113-1, intracellular staining). FIG. 4C. Graphical representation of flow cytometric data showing the number of transplanted cells with an injection of a different number of cells per mouse heart. No cells, 125 μL of PBS was injected; K, ×10³. n=5 per group; One-Way ANOVA, Tukey post-test. FIG. 4D. The percentage of mouse CD31 endothelial cells and transplanted human HiPC (stained with antibody against human CD29/β1-integrin) in sham-operated animals (sham, left plots), mice injected with 25 μL of PBS (middle) or 2.5×10⁵ hHiPC cells (in 25 μl of PBS, right plots). Red gate showing CD29 expressing human HiPC. Upper panel—Day 1 (24 hours) after MI, lower panel—day 5 after MI. FIG. 4E. The number of HiPC cells was calculated using a total number of viable cells and the percent of human hHiPC cells expressing human CD29. n=18, day 1, n=12, day 5. Unpaired t test. Horizontal line shows mean value of number of transplanted hHiPC. (FIGS. 4F-4I) Flow cytometric histogram of cardiac cell suspension obtained on day 5 post-MI and demonstrating (FIG. 4F) relative cell sizes using forward scatter (FSC), which were analyzed by determining (FIG. 4G) median values of forward scatter area (FSC-A), and (FIG. 411) morphological characteristics of cell complexity using side scatter, analyzed after examination of (FIG. 41) median values of side scatter area (SSC-A) of mouse CD29 positive cells (Mouse, gray), human transplanted HiPC (t-HiPC, orange) and cultured human HiPC (c-HiPC, blue) before intramyocardial injection. (FIG. 4G and FIG. 41) n=12, one-way ANOVA, Tukey's posttest. (FIGS. 4J-4K) Graphical representation of flow cytometric data showing expression of (FIG. 4J) CD29 and (FIG. 4K) CD105 on the surface of hHiPCs in culture (c-HiPC) before intramyocardial delivery, and HiPC cell obtained from post-MI (day 5) mouse heart (t-HiPC). n=12, unpaired t test.

FIGS. 5A-5E. hHiPC treatment following myocardial infarction improves heart function. Mice were randomized to receive control PBS or hHiPC treatment. 250,000 hHiPCs (n=4 clones pooled) or PBS were injected into the peri-infarct area of NSG male and female mice (n=37) immediately after ligation of LCA. Fractional shortening (FS) was analyzed before MI (baseline) and on days 7, 14, 21, and 28 post-MI. FIG. 5A. FS were improved in hHiPC treated mice 28 days post-MI compared to PBS controls. Data were analyzed by two-way ANOVA repeated measures with Sidak's multiple comparison test. *=p<0.05, **p<0.01 hHiPC vs PBS; ††=p<0.01 PBS day 7 vs PBS day 28; ‘ns’=p>0.05. FIG. 5B. Survival data were analyzed using Kaplan-Meier curve and shows a trend towards improved survival of male mice (circle) following hHiPC injection (red) compared to PBS control (blue). No effect of hHiPC injection was found in female mice (triangle) due to high survivability in both groups (90-100%). P-value determined using Mantel-Cox test; *Male mice with PBS vs. hHiPC injections; †female mice with PBS vs. hHiPC injections. FIG. 5C. Echocardiographic parameters of heart weight/body weight (HW/BW), left ventricle (LV) mass, heart rate, LV anterior wall thickness; diastole (LVAWd) and systole (LVAWs), LV posterior wall thicknesses; diastole (LVPWd) and systole (LVPWs), LV internal diameter; end diastole (LVIDd) and end systole (LVIDs), were analyzed at 28-days post-MI using unpaired t test. Values expressed as mean±SEM. FIG. 5D Masson trichrome staining of mouse hearts isolated on day 28 post-MI from mice that received PBS (left) or hHiPCs (right); original magnification, ×4. FIG. 5E Quantification of scar size. n=16 per group, unpaired t test.

FIG. 6. Variability in the cardiac function after intramyocardial injection of different clones. Experimental myocardial infarction was induced in 15-16 weeks-old mice. A total of 2.5×10⁵ HiPC cells per heart (four clones, each from a different individual) were injected into the peri-infarct area approximately 10 minutes later after MI. Cells for injections were prepared in parallel and grown in similar conditions for two passages (P2). The differences in the injected animals are due to the difference in cell availability (cells obtained from different clones are characterized by different growth dynamics). Number of injected animals: PBS, n=11; Clone 11, n=3; Clone 24, n=4; Clone 03, n=6; Clone 48, n=7. P values were calculated using RM two-way ANOVA, Tukey's multiple post-test.

FIGS. 7A-7B. The intramyocardial transplantation of hHiPC induce accumulation of CD45 immune cells compared to PBS control. Experimental myocardial infarction was induced in 15-16 weeks-old mice. A total of 2.5×10⁵ HiPC cells per heart (four clones, each from a different individual) were injected into the peri-infarct area approximately 10 minutes later after MI. Injection of the same volume of PBS (25 pl) was used as a vehicle control for HiPC. FIG. 7A. Graphical representation of flow cytometry data showing the total number of cardiac non-myocyte cells (left), the number of non-immune cells (middle) and CD45-positive immune cells (right) in sham-operated animals (sham) and animals injected with PBS or HiPC for 24 hours after experimental myocardial infarction. FIG. 7B. The total number of cardiac non-myocyte cells (left), the number of non-immune cells (middle), and CD45-positive immune cells (right) in sham-operated animals (sham) and animals injected with PBS or HiPC for 5 days after experimental myocardial infarction. Number of animals: Sham, n=7; PBS, n=8; HiPC, n=18. P values were calculated using ordinary one-way ANOVA, Tukey's multiple post-test.

FIGS. 8A-8C. Transplanted hHiPC promotes accumulation of granulocytic phagocytes compared to PBS control. Experimental myocardial infarction was induced in 15-16 weeks-old mice. A total of 2.5×10⁵ HiPC cells per heart (four clones, each from a different individual) were injected into the peri-infarct area approximately 10 minutes later after MI. Injection of the same volume of PBS (25 μl) was used as a vehicle control for HiPC. FIG. 8A. Representative flow cytometric plots demonstrating the strategy to identify CD11b+Ly6G+ granulocytic phagocytes in sham-operated animals (Sham) and on 24 hr and 5 days (120 hr) after experimental myocardial injury. FIGS. 8B-8C. Graphical representation of flow cytometric data showing the number of granulocytic phagocytes in Sham, PBS, and HiPC groups on (FIG. 8B) 24 hr and (FIG. 8C) 5 days (120 hr) after experimental myocardial injury Number of animals: Sham, n=7; PBS, n=8; HiPC, n=18. P values were calculated using ordinary one-way ANOVA, Tukey's multiple post-test.

FIG. 9. Mice with overexpression of CTHRC1 in fibroblasts demonstrated improved cardiac function. Experimental myocardial infarction was induced in 15-16 weeks-old wilt-type mice (Wild type, red, control) and animals with overexpression of CTHRC1 in cardiac fibroblasts (CTHRC1 overexpressors, blue). Cardiac function were monitored using echocardiography for one month after the induction of MI. Wild type, n=16 (50/50 male/female), CTHRC1 overexpressors, n=15 (50/50, male/female). Kruskal Wallis test, Dunn's post-test

### DETAILED DESCRIPTION

Cardiac-derived human highly proliferative cells (hHiPCs) were determined to have unique novel properties to survive over the acute inflammatory phase in the ischemic myocardium. hHIPCs were identified as potential candidates for cell-based therapy to create a long-term prohealing microenvironment by secreting proreparative proteins CXCL6, CTHRC1, and CD73.

hHiPCs are a highly proliferative expandable cell population isolated from the human ventricular myocardium.² hHiPCs can be expanded from single-cell suspensions and have strong surface expression of CD105 (endoglin), a human mesenchymal stem cell marker,^{3,4} which plays a role in therapeutic cell repair and paracrine-mediated angiogenesis,^{5,6} and CD29, a marker of therapeutic stem cells.^{2,7,8} In contrast, hHiPCs do not express the pan-immune cell marker CD45 or the cardiac progenitor cell/cardiosphere-derived cell (CDC) marker, CD 117 (c-Kit), thus defining hHiPCs as a distinct population of mesenchymal cells with progenitor and proangiogenic capabilities.⁹ Furthermore, signaling through the CD105 coreceptor, ALK1 (ACVRL1), and its ligand, BMP9, regulates proangiogenic paracrine and autocrine responses through regulation of SOST (sclerostin), ISLR (meflin), and IGFBP3 in hHiPCs. 10 hHiPCs can differentiate toward the endothelial cell (EC) lineage in vitro, which may contribute to myocardial repair at the site of infarction. With their innate ability to form colonies from single-cell isolates, hHiPCs are capable of >50 population doublings while maintaining phenotypic stability and characteristic surface marker expression.² A deeper understanding of hHiPC ability to promote repair in vivo is warranted to better understand their potential role in myocardial recovery and prevention of heart failure.

### Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to the subject.

The term “isolated” refers to a material that is substantially or essentially free from components, which are used to produce the material. The lower end of the range of purity for the polymer networks is about 60%, about 70% or about 80% and the upper end of the range of purity is about 70%, about 80%, about 90% or more than about 90%.

The term “autologous cells”, as used herein, refers to cells which are person's own genetically identical cells.

The term “heterologous cells”, as used herein, refers to cells which are not person's own and are genetically different cells.

The term “plurality” is used herein to refer to the state of being plural, i.e., at least two, e.g., cells, e.g., a plurality of highly proliferative cells. Plurality can mean the cells in the plurality of cells can be homogenous or heterogeneous, e.g., homogeneous cells can derived from a signal clone or colony, or heterogeneous cells can be derived from multiple colonies.

The terms “subject,” “patient,” “host” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

By “treatment,” “treating,” or “treat” is meant that at least an amelioration of the symptoms associated with the condition afflicting the host is achieved, where amelioration is used in a broad sense to refer to at least a reduction in the magnitude of a parameter, e.g. symptom (such as ejection fraction, Starling relationship, regional ventricular wall strains, infarct extension, expansion of the border zone wall, border zone stress amplitude) associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

### List of Abbreviations

LCA; Left Coronary Artery;
MI; Myocardial Infarction;
CM, Conditioned Media;
hHiPC, Human Highly Proliferative Cells;
HREC, Human Retinal Endothelial Cells;
CDC, Cardiosphere Derived Cells;
DTT, Dithiothreitol;
IAA, Iodoacetamide;
LC-MS/MS, Liquid Chromatography with tandem mass spectrometry;
SWATH, Sequential Windowing of All Theoretical Mass Spectra;

### Compositions of Highly Proliferative Cells

According to the present disclosure, compositions are provided for herein which can include a plurality of highly proliferative cells, wherein the plurality of highly proliferative cells express one or more of CXCL6, CTHRC1, and CD73, and wherein the highly proliferative cells are generated by clonal expansion of a cell.

The cell comprises a stem cell or a differentiated cell. In some embodiments, the stem cell can be an induced pluripotent stem cell (iPSC), or the differentiated cell can be a heart cell. The cell can be derived from pluripotent cell, such as an induced pluripotent stem cell (iPSC). The cell can be a cardiac stem cell.

The heart cell can be selected from the group consisting of an epicardial cell, a pericardial cell, a cardiomyocyte, a cardiac endothelial cell, a cardiac fibroblast cell, and an endocardial cell. The heart cell can be derived from the same subject which will be administered the composition as disclosed herein. The heart cell can be derived from another subject which will be administered the composition as disclosed herein, the heart cell can be derived from a biopsy of a subject's heart, such as but not limited to the left ventricle, right ventricle, left atrium, right atrium, septum, atrioventricular valves, semilunar valves, coronary arteries and veins, ventricle epicardial tissues, and pericardium.

### Highly Proliferative Cells

The plurality of highly proliferative cells can express one or more of 1B46 (oligo peptide binding protein), CREG1 (Cellular Repressor of ElA-stimulated Genes 1), MRCl (Mannose Receptor C-type 1), TFPI2 (Tissue Factor Pathway Inhibitor 2), CD200 (aka OX2), PLXB2 (Plexin-B2), or LRP1 (Low-Density Lipoprotein Receptor-Related Protein 1). The plurality of highly proliferative cells can express one or more of ACTN4 (Alpha-actinin-4), APPL1 (Adaptor protein, phosphotyrosine interacting with PH domain and leucine zipper 1), CAPZA1 (capping actin protein of muscle Z-line alpha subunit 1), CNN2 (Calponin 2), COL1A2 (Collagen alpha-2(I) chain), HIST2H3D (a histone H3 variant), ISG15 (Interferon-Stimulated Gene 15), MAOA (Monoamine Oxidase A), MTAP (Methylthioadenosine Phosphorylase), MX1 (Myxovirus resistance protein 1), PSMA2 (Proteasome Subunit Alpha Type-2), PSMB4 (Proteasome Subunit Beta Type-4), PSMB7 (Proteasome Subunit Beta Type-7), PSMB9 (Proteasome Subunit Beta Type-9), PSME1 (Proteasome activator complex subunit 1), PYCARD (aka Apoptosis-associated speck-like protein containing a CARD), RIPK2 (Receptor-Interacting Protein Kinase 2), STAT1 (Signal Transducer and Activator of Transcription 1), STAT2 (Signal Transducer and Activator of Transcription 2), or YAP (Yes-associated protein 1). The plurality of highly proliferative cells can express one or more of CXCL5 (C-X-C motif chemokine ligand 5), CXCL1 (C-X-C motif chemokine ligand 1), CXCL3 (C-X-C motif chemokine ligand 3), CXCL8 (Interleukin-8 or IL-8), AGRIN, or CO3 (Complement C3).

In some embodiments, the plurality of highly proliferative cells exhibit upregulated and downregulated genes when compared to an endothelial cell. In some embodiments, the endothelial cell can be terminally differentiated. The plurality of highly proliferative cells can upregulate one or more of CO3A1 (alpha-1 chain of Type III Collagen), CXCL6 (C-X-C motif chemokine 6), AK1C3, H11, HLA-B, AFAP1 (Actin filament-associated protein 1), TAGLN (transgelin), TYPH (Thymidine phosphorylase), RL15, ST1A1, CREG1, PDCD6, HEXA, ECHD1, MYOSA, CHM2B, CO1A1 (collagen alpha-1(I) chain), EWS (Ewing Sarcoma), MMSA (Merozoite Surface Antigen), and/or SERA compared to endothelial cells. The plurality of highly proliferative cells can downregulate one or more of RS11 (Ribosomal Protein S11), ICAM2 (intercellular adhesion molecule 2), R39L5 (Ribosomal Protein L39-Like), IF2B1 (Insulin-like growth factor 2 mRNA-binding protein 1), EPCR (Endothelial protein C receptor), RS4Y1 (Small ribosomal subunit protein eS4, Y isoform 1), SYDC, VWF (von Willebrand factor), H2B1M (histone H2B type 1-M), and/or ACTG (gamma-actin (γ-actin)).

The plurality of highly proliferative cells can secrete one or more proteins, such as cytokines. The plurality of highly proliferative cells can secrete one or more of FINC (fibronectin), PGBM (basement membrane-specific heparan sulfate proteoglycan core protein), CO3, C06A3 (Collagen alpha-3(VI) chain), FBN1 (Fibrillin-1), CO1A2 (Collagen alpha-2(I) chain), TSP1 (Thrombospondin-1), CO1A1, CFAH (Complement factor H), POSTN (Periostin), MMP2 (72 kDa type IV collagenase), FLNA (Filamin-A), ACTN4 (Alpha-actinin-4), TSP2 (Thrombospondin-2), AGRIN, BGH3 (Transforming growth factor-beta-induced protein ig-h3), LTBP2 (Latent-transforming growth factor beta-binding protein 2), PXDN (Peroxidasin homolog), C06A1 (Collagen alpha-1(VI) chain), PAPP1 (Pappalysin-1), PAIl (Plasminogen activator inhibitor 1), C1R (Complement Clr subcomponent), KPYM (Pyruvate kinase PKM), VIME (Vimentin), and/or HSP7C (Heat shock cognate 71 kDa protein).

The plurality of highly proliferative cells do not express c-kit and/or CD34. The composition of plurality of highly proliferative cells does not include CD45+ cells.

Highly proliferative cells can be larger in size than most cells. In some embodiments, the highly proliferative cells can be between about 25 μm to about 110 μm in size. In some embodiments, the highly proliferative cells can be between about 25 μm to about 100 μm, about μm to about 100 μm, about 35 μm to about 100 μm, about 40 μm to about 100 μm, about 45 μm to about 100 μm, about 50 μm to about 100 μm, about 55 μm to about 100 μm, about 60 μm to about 100 μm, about 65 μm to about 100 μm, about 70 μm to about 100 μm, about 75 μm to about 100 μm, about 80 μm to about 100 μm, about 85 μm to about 100 μm, about 90 μm to about 100 μm, about 95 μm to about 100 μm, about 25 μm to about 95 μm, about 30 μm to about 95 μm, about 35 μm to about 95 μm, about 40 μm to about 95 μm, about 45 μm to about 95 μm, about 50 μm to about 95 μm, about 55 μm to about 95 μm, about 60 μm to about 95 μm, about 65 μm to about 95 μm, about 70 μm to about 95 μm, about 75 μm to about 95 μm, about 80 μm to about 95 μm, about 85 μm to about 95 μm, about 90 μm to about 95 μm, about 25 μm to about 90 μm, about 30 μm to about 90 μm, about 35 μm to about 90 μm, about 40 μm to about 90 μm, about 45 μm to about 90 μm, about 50 μm to about 90 μm, about 55 μm to about 90 μm, about 60 μm to about 90 μm, about 65 μm to about 90 μm, about 70 μm to about 90 μm, about 75 μm to about 90 μm, about 80 μm to about 90 μm, about 85 μm to about 90 μm, about 25 μm to about 85 μm, about 30 μm to about 85 μm, about 35 μm to about 85 μm, about 40 μm to about 85 μm, about 45 μm to about 85 μm, about 50 μm to about 85 μm, about 55 μm to about 85 μm, about 60 μm to about 85 μm, about 65 μm to about 85 μm, about 70 μm to about 85 μm, about 75 μm to about 85 μm, about 80 μm to about 85 μm, about 25 μm to about 80 μm, about 30 μm to about 80 μm, about 35 μm to about 80 μm, about 40 μm to about 80 μm, about 45 μm to about 80 μm, about 50 μm to about 80 μm, about 55 μm to about 80 μm, about 60 μm to about 80 μm, about 65 μm to about 80 μm, about 70 μm to about 80 μm, about 75 μm to about 80 μm, about 25 μm to about 75 μm, about 30 μm to about 75 μm, about 35 μm to about 75 μm, about 40 μm to about 75 μm, about 45 μm to about 75 μm, about 50 μm to about 75 μm, about 55 μm to about 75 μm, about 60 μm to about 75 μm, about 65 μm to about 75 μm, about 70 μm to about 75 μm, about m to about 70 μm, about 30 μm to about 70 μm, about 35 μm to about 70 μm, about 40 μm to about 70 μm, about 45 μm to about 70 μm, about 50 μm to about 70 μm, about 55 μm to about 70 μm, about 60 μm to about 70 μm, about 65 μm to about 70 μm, about 25 μm to about 65 μm, about 30 μm to about 65 μm, about 35 μm to about 65 μm, about 40 μm to about 65 μm, about m to about 65 μm, about 50 μm to about 65 μm, about 55 μm to about 65 μm, about 60 μm to about 65 μm, about 25 μm to about 60 μm, about 30 μm to about 60 μm, about 35 μm to about 60 μm, about 40 μm to about 60 μm, about 45 μm to about 60 μm, about 50 μm to about 60 μm, about 55 μm to about 60 μm, about 25 μm to about 55 μm, about 30 μm to about 55 μm, about μm to about 55 μm, about 40 μm to about 55 μm, about 45 μm to about 55 μm, about 50 μm to about 55 μm, about 25 μm to about 50 μm, about 30 μm to about 50 μm, about 35 μm to about 50 μm, about 40 μm to about 50 μm, about 45 μm to about 50 μm, about 25 μm to about 45 μm, about 30 μm to about 45 μm, about 35 μm to about 45 μm, about 40 μm to about 45 μm, about m to about 40 μm, about 30 μm to about 40 μm, about 35 μm to about 40 μm, about 25 μm to about 35 μm, about 30 μm to about 35 μm, or about 25 μm to about 30 μm in size. A preferred cell size is between approximately about 50 μm and about 80 μm.

The concentration of the a culture of the highly proliferative cells can increase at least by 2-fold in about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, or about 90 hours.

The concentration of the a culture of the highly proliferative cells can increase at least by 2-fold in at least about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, or about 90 hours.

### Pharmaceutical Composition

In some embodiments, a pharmaceutical composition comprises the composition as described herein and a pharmaceutically acceptable carrier. The pharmaceutical carrier can maintain and stabilize the highly proliferative cells of the composition for administration to a subject. The pharmaceutically acceptable excipient can suitable for, but is not limited to intracardiac injection.

### Methods of Producing a Composition

The composition including a plurality of highly proliferative cells can be produced by providing a cell; culturing the cell to form a colony. The cells of the colony can be between about 50 μm to about 110 μm in size, and wherein a concentration of a culture of the cells of the colony increase by at least 2-fold in about 15 hours to about 90 hours.

The cell can be a stem cell or a differentiated cell. In some embodiments, the stem cell can be an induced pluripotent stem cell (iPSC), or the differentiated cell can be a heart cell.

The heart cell can be selected from the group consisting of an epicardial cell, a pericardial cell, a cardiomyocyte, a cardiac endothelial cell, a cardiac fibroblast cell, and an endocardial cell. The heart cell can be derived from the same subject which will be administered the composition as disclosed herein. The heart cell can be derived from another subject which will be administered the composition as disclosed herein. The heart cell can be derived from a biopsy of a subject's heart, such as but not limited to the left ventricle, right ventricle, left atrium, right atrium, septum, atrioventricular valves, semilunar valves, coronary arteries and veins, and pericardium.

The heart cell can be derived from pluripotent cell, such as an induced pluripotent stem cell (iPSC). The heart cell can be a cardiac stem cell.

In some embodiments, the cell can be cultured in a medium including fetal bovine serum (FBS) and an endothelial cell growth media. The media can include additives, such as, but not limited to growth factors, cytokines, antibiotics, and nutrients. The media can include Medium 199 (M199) and Endothelial Cell Growth Medium-2 (EGM-2).

The composition can also be depleted of CD45+ cells. For example, anti-CD45 antibodies can be used to bind and remove CD45+ cells. The anti-CD45 antibodies can be conjugated on a surface, such as a magnetic surface, e.g., magnetic beads. In some embodiments, CD45+ cells can be removed by density gradient centrifugation or flow cytometry.

In some embodiments, the composition including a plurality of highly proliferative cells can comprise between about 2×10⁴ cells to about 5.5×10⁵ cells. The composition including a plurality of highly proliferative cells can comprise between about 2×10⁴ to about 5.5×10⁵ cells, about 2.5×10⁴ to about 5.5×10⁵ cells, about 3×10⁴ to about 5.5×10⁵ cells, about 3.5×10⁴ to about 5.5×10⁵ cells, about 4×10⁴ to about 5.5×10⁵ cells, about 4.5×10⁴ to about 5.5×10⁵ cells, about 5×10⁴ to about 5.5×10⁵ cells, about 5.5×10⁴ to about 5.5×10⁵ cells, about 6×10⁴ to about 5.5×10⁵ cells, about 6.5×10⁴ to about 5.5×10⁵ cells, about 7×10⁴ to about 5.5×10⁵ cells, about 7.5×10⁴ to about 5.5×10⁵ cells, about 8×10⁴ to about 5.5×10⁵ cells, about 8.5×10⁴ to about 5.5×10⁵ cells, about 9×10⁴ to about 5.5×10⁵ cells, about 9.5×10⁴ to about 5.5×10⁵ cells, about 1×10⁵ to about 5.5×10⁵ cells, about 1.5×10⁵ to about 5.5×10⁵ cells, about 2×10⁵ to about 5.5×10⁵ cells, about 2.5×10⁵ to about 5.5×10⁵ cells, about 3×10⁵ to about 5.5×10⁵ cells, about 3.5×10⁵ to about 5.5×10⁵ cells, about 4×10⁵ to about 5.5×10⁵ cells, about 4.5×10⁵ to about 5.5×10⁵ cells, about 5×10⁵ to about 5.5×10⁵ cells, about 2×10⁴ to about 5×10⁵ cells, about 2.5×10⁴ to about 5×10⁵ cells, about 3×10⁴ to about 5×10⁵ cells, about 3.5×10⁴ to about 5×10⁵ cells, about 4×10⁴ to about 5×10⁵ cells, about 4.5×10⁴ to about 5×10⁵ cells, about 5×10⁴ to about 5×10⁵ cells, about 5.5×10⁴ to about 5×10⁵ cells, about 6×10⁴ to about 5×10⁵ cells, about 6.5×10⁴ to about 5×10⁵ cells, about 7×10⁴ to about 5×10⁵ cells, about 7.5×10⁴ to about 5×10⁵ cells, about 8×10⁴ to about 5×10⁵ cells, about 8.5×10⁴ to about 5×10⁵ cells, about 9×10⁴ to about 5×10⁵ cells, about 9.5×10⁴ to about 5×10⁵ cells, about 1×10⁵ to about 5×10⁵ cells, about 1.5×10⁵ to about 5×10⁵ cells, about 2×10⁵ to about 5×10⁵ cells, about 2.5×10⁵ to about 5×10⁵ cells, about 3×10⁵ to about 5×10⁵ cells, about 3.5×10⁵ to about 5×10⁵ cells, about 4×10⁵ to about 5×10⁵ cells, about 4.5×10⁵ to about 5×10⁵ cells, about 2×10⁴ to about 4.5×10⁵ cells, about 2.5×10⁴ to about 4.5×10⁵ cells, about 3×10⁴ to about 4.5×10⁵ cells, about 3.5×10⁴ to about 4.5×10⁵ cells, about 4×10⁴ to about 4.5×10⁵ cells, about 4.5×10⁴ to about 4.5×10⁵ cells, about 5×10⁴ to about 4.5×10⁵ cells, about 5.5×10⁴ to about 4.5×10⁵ cells, about 6×10⁴ to about 4.5×10⁵ cells, about 6.5×10⁴ to about 4.5×10⁵ cells, about 7×10⁴ to about 4.5×10⁵ cells, about 7.5×10⁴ to about 4.5×10⁵ cells, about 8×10⁴ to about 4.5×10⁵ cells, about 8.5×10⁴ to about 4.5×10⁵ cells, about 9×10⁴ to about 4.5×10⁵ cells, about 9.5×10⁴ to about 4.5×10⁵ cells, about 1×10⁵ to about 4.5×10⁵ cells, about 1.5×10⁵ to about 4.5×10⁵ cells, about 2×10⁵ to about 4.5×10⁵ cells, about 2.5×10⁵ to about 4.5×10⁵ cells, about 3×10⁵ to about 4.5×10⁵ cells, about 3.5×10⁵ to about 4.5×10⁵ cells, about 4×10⁵ to about 4.5×10⁵ cells, about 2×10⁴ to about 4×10⁵ cells, about 2.5×10⁴ to about 4×10⁵ cells, about 3×10⁴ to about 4×10⁵ cells, about 3.5×10⁴ to about 4×10⁵ cells, about 4×10⁴ to about 4×10⁵ cells, about 4.5×10⁴ to about 4×10⁵ cells, about 5×10⁴ to about 4×10⁵ cells, about 5.5×10⁴ to about 4×10⁵ cells, about 6×10⁴ to about 4×10⁵ cells, about 6.5×10⁴ to about 4×10⁵ cells, about 7×10⁴ to about 4×10⁵ cells, about 7.5×10⁴ to about 4×10⁵ cells, about 8×10⁴ to about 4×10⁵ cells, about 8.5×10⁴ to about 4×10⁵ cells, about 9×10⁴ to about 4×10⁵ cells, about 9.5×10⁴ to about 4×10⁵ cells, about 1×10⁵ to about 4×10⁵ cells, about 1.5×10⁵ to about 4×10⁵ cells, about 2×10⁵ to about 4×10⁵ cells, about 2.5×10⁵ to about 4×10⁵ cells, about 3×10⁵ to about 4×10⁵ cells, about 3.5×10⁵ to about 4×10⁵ cells, about 2×10⁴ to about 3.5×10⁵ cells, about 2.5×10⁴ to about 3.5×10⁵ cells, about 3×10⁴ to about 3.5×10⁵ cells, about 3.5×10⁴ to about 3.5×10⁵ cells, about 4×10⁴ to about 3.5×10⁵ cells, about 4.5×10⁴ to about 3.5×10⁵ cells, about 5×10⁴ to about 3.5×10⁵ cells, about 5.5×10⁴ to about 3.5×10⁵ cells, about 6×10⁴ to about 3.5×10⁵ cells, about 6.5×10⁴ to about 3.5×10⁵ cells, about 7×10⁴ to about 3.5×10⁵ cells, about 7.5×10⁴ to about 3.5×10⁵ cells, about 8×10⁴ to about 3.5×10⁵ cells, about 8.5×10⁴ to about 3.5×10⁵ cells, about 9×10⁴ to about 3.5×10⁵ cells, about 9.5×10⁴ to about 3.5×10⁵ cells, about 1×10⁵ to about 3.5×10⁵ cells, about 1.5×10⁵ to about 3.5×10⁵ cells, about 2×10⁵ to about 3.5×10⁵ cells, about 2.5×10⁵ to about 3.5×10⁵ cells, about 3×10⁵ to about 3.5×10⁵ cells, about 2×10⁴ to about 3×10⁵ cells, about 2.5×10⁴ to about 3×10⁵ cells, about 3×10⁴ to about 3×10⁵ cells, about 3.5×10⁴ to about 3×10⁵ cells, about 4×10⁴ to about 3×10⁵ cells, about 4.5×10⁴ to about 3×10⁵ cells, about 5×10⁵ to about 3×10⁵ cells, about 5.5×10⁴ to about 3×10⁵ cells, about 6×10⁴ to about 3×10⁵ cells, about 6.5×10⁴ to about 3×10⁵ cells, about 7×10⁴ to about 3×10⁵ cells, about 7.5×10⁴ to about 3×10⁵ cells, about 8×10⁴ to about 3×10⁵ cells, about 8.5×10⁴ to about 3×10⁵ cells, about 9×10⁴ to about 3×10⁵ cells, about 9.5×10⁴ to about 3×10⁵ cells, about 1×10⁵ to about 3×10⁵ cells, about 1.5×10⁵ to about 3×10⁵ cells, about 2×10⁵ to about 3×10⁵ cells, about 2.5×10⁵ to about 3×10⁵ cells, about 2×10⁴ to about 2.5×10⁵ cells, about 2.5×10⁴ to about 2.5×10⁵ cells, about 3×10⁴ to about 2.5×10⁵ cells, about 3.5×10⁴ to about 2.5×10⁵ cells, about 4×10⁴ to about 2.5×10⁵ cells, about 4.5×10⁴ to about 2.5×10⁵ cells, about 5×10⁴ to about 2.5×10⁵ cells, about 5.5×10⁴ to about 2.5×10⁵ cells, about 6×10⁴ to about 2.5×10⁵ cells, about 6.5×10⁴ to about 2.5×10⁵ cells, about 7×10⁴ to about 2.5×10⁵ cells, about 7.5×10⁴ to about 2.5×10⁵ cells, about 8×10⁴ to about 2.5×10⁵ cells, about 8.5×10⁴ to about 2.5×10⁵ cells, about 9×10⁴ to about 2.5×10⁵ cells, about 9.5×10⁴ to about 2.5×10⁵ cells, about 1×10⁵ to about 2.5×10⁵ cells, about 1.5×10⁵ to about 2.5×10⁵ cells, about 2×10⁵ to about 2.5×10⁵ cells, about 2×10⁴ to about 2×10⁵ cells, about 2.5×10⁴ to about 2×10⁵ cells, about 3×10⁴ to about 2×10⁵ cells, about 3.5×10⁴ to about 2×10⁵ cells, about 4×10⁴ to about 2×10⁵ cells, about 4.5×10⁴ to about 2×10⁵ cells, about 5×10⁴ to about 2×10⁵ cells, about 5.5×10⁴ to about 2×10⁵ cells, about 6×10⁴ to about 2×10⁵ cells, about 6.5×10⁴ to about 2×10⁵ cells, about 7×10⁴ to about 2×10⁵ cells, about 7.5×10⁴ to about 2×10⁵ cells, about 8×10⁴ to about 2×10⁵ cells, about 8.5×10⁴ to about 2×10⁵ cells, about 9×10⁴ to about 2×10⁵ cells, about 9.5×10⁴ to about 2×10⁵ cells, about 1×10⁵ to about 2×10⁵ cells, about 1.5×10⁵ to about 2×10⁵ cells, about 2×10⁴ to about 1.5×10⁵ cells, about 2.5×10⁴ to about 1.5×10⁵ cells, about 3×10⁴ to about 1.5×10⁵ cells, about 3.5×10⁴ to about 1.5×10⁵ cells, about 4×10⁴ to about 1.5×10⁵ cells, about 4.5×10⁴ to about 1.5×10⁵ cells, about 5×10⁴ to about 1.5×10⁵ cells, about 5.5×10⁴ to about 1.5×10⁵ cells, about 6×10⁴ to about 1.5×10⁵ cells, about 6.5×10⁴ to about 1.5×10⁵ cells, about 7×10⁴ to about 1.5×10⁵ cells, about 7.5×10⁴ to about 1.5×10⁵ cells, about 8×10⁴ to about 1.5×10⁵ cells, about 8.5×10⁴ to about 1.5×10⁵ cells, about 9×10⁴ to about 1.5×10⁵ cells, about 9.5×10⁴ to about 1.5×10⁵ cells, about 1×10⁵ to about 1.5×10⁵ cells, about 2×10⁴ to about 1×10⁵ cells, about 2.5×10⁴ to about 1×10⁵ cells, about 3×10⁴ to about 1×10⁵ cells, about 3.5×10⁴ to about 1×10⁵ cells, about 4×10⁴ to about 1×10⁵ cells, about 4.5×10⁴ to about 1×10⁵ cells, about 5×10⁴ to about 1×10⁵ cells, about 5.5×10⁴ to about 1×10⁵ cells, about 6×10⁴ to about 1×10⁵ cells, about 6.5×10⁴ to about 1×10⁵ cells, about 7×10⁴ to about 1×10⁵ cells, about 7.5×10⁴ to about 1×10⁵ cells, about 8×10⁴ to about 1×10⁵ cells, about 8.5×10⁴ to about 1×10⁵ cells, about 9×10⁴ to about 1×10⁵ cells, about 9.5×10⁴ to about 1×10⁵ cells, about 2×10⁴ to about 9.5×10⁴ cells, about 2.5×10⁴ to about 9.5×10⁴ cells, about 3×10⁴ to about 9.5×10⁴ cells, about 3.5×10⁴ to about 9.5×10⁴ cells, about 4×10⁴ to about 9.5×10⁴ cells, about 4.5×10⁴ to about 9.5×10⁴ cells, about 5×10⁴ to about 9.5×10⁴ cells, about 5.5×10⁴ to about 9.5×10⁴ cells, about 6×10⁴ to about 9.5×10⁴ cells, about 6.5×10⁴ to about 9.5×10⁴ cells, about 7×10⁴ to about 9.5×10⁴ cells, about 7.5×10⁴ to about 9.5×10⁴ cells, about 8×10⁴ to about 9.5×10⁴ cells, about 8.5×10⁴ to about 9.5×10⁴ cells, about 9×10⁴ to about 9.5×10⁴ cells, about 2×10⁴ to about 9×10⁴ cells, about 2.5×10⁴ to about 9×10⁴ cells, about 3×10⁴ to about 9×10⁴ cells, about 3.5×10⁴ to about 9×10⁴ cells, about 4×10⁴ to about 9×10⁴ cells, about 4.5×10⁴ to about 9×10⁴ cells, about 5×10⁴ to about 9×10⁴ cells, about 5.5×10⁴ to about 9×10⁴ cells, about 6×10⁴ to about 9×10⁴ cells, about 6.5×10⁴ to about 9×10⁴ cells, about 7×10⁴ to about 9×10⁴ cells, about 7.5×10⁴ to about 9×10⁴ cells, about 8×10⁴ to about 9×10⁴ cells, about 8.5×10⁴ to about 9×10⁴ cells, about 2×10⁴ to about 8.5×10⁴ cells, about 2.5×10⁴ to about 8.5×10⁴ cells, about 3×10⁴ to about 8.5×10⁴ cells, about 3.5×10⁴ to about 8.5×10⁴ cells, about 4×10⁴ to about 8.5×10⁴ cells, about 4.5×10⁴ to about 8.5×10⁴ cells, about 5×10⁴ to about 8.5×10⁴ cells, about 5.5×10⁴ to about 8.5×10⁴ cells, about 6×10⁴ to about 8.5×10⁴ cells, about 6.5×10⁴ to about 8.5×10⁴ cells, about 7×10⁴ to about 8.5×10⁴ cells, about 7.5×10⁴ to about 8.5×10⁴ cells, about 8×10⁴ to about 8.5×10⁴ cells, about 2×10⁴ to about 8×10⁴ cells, about 2.5×10⁴ to about 8×10⁴ cells, about 3×10⁴ to about 8×10⁴ cells, about 3.5×10⁴ to about 8×10⁴ cells, about 4×10⁴ to about 8×10⁴ cells, about 4.5×10⁴ to about 8×10⁴ cells, about 5×10⁴ to about 8×10⁴ cells, about 5.5×10⁴ to about 8×10⁴ cells, about 6×10⁴ to about 8×10⁴ cells, about 6.5×10⁴ to about 8×10⁴ cells, about 7×10⁴ to about 8×10⁴ cells, about 7.5×10⁴ to about 8×10⁴ cells, about 2×10⁴ to about 7.5×10⁴ cells, about 2.5×10⁴ to about 7.5×10⁴ cells, about 3×10⁴ to about 7.5×10⁴ cells, about 3.5×10⁴ to about 7.5×10⁴ cells, about 4×10⁴ to about 7.5×10⁴ cells, about 4.5×10⁴ to about 7.5×10⁴ cells, about 5×10⁴ to about 7.5×10⁴ cells, about 5.5×10⁴ to about 7.5×10⁴ cells, about 6×10⁴ to about 7.5×10⁴ cells, about 6.5×10⁴ to about 7.5×10⁴ cells, about 7×10⁴ to about 7.5×10⁴ cells, about 2×10⁴ to about 7×10⁴ cells, about 2.5×10⁴ to about 7×10⁴ cells, about 3×10⁴ to about 7×10⁴ cells, about 3.5×10⁴ to about 7×10⁴ cells, about 4×10⁴ to about 7×10⁴ cells, about 4.5×10⁴ to about 7×10⁴ cells, about 5×10⁴ to about 7×10⁴ cells, about 5.5×10⁴ to about 7×10⁴ cells, about 6×10⁴ to about 7×10⁴ cells, about 6.5×10⁴ to about 7×10⁴ cells, about 2×10⁴ to about 6.5×10⁴ cells, about 2.5×10⁴ to about 6.5×10⁴ cells, about 3×10⁴ to about 6.5×10⁴ cells, about 3.5×10⁴ to about 6.5×10⁴ cells, about 4×10⁴ to about 6.5×10⁴ cells, about 4.5×10⁴ to about 6.5×10⁴ cells, about 5×10⁴ to about 6.5×10⁴ cells, about 5.5×10⁴ to about 6.5×10⁴ cells, about 6×10⁴ to about 6.5×10⁴ cells, about 2×10⁴ to about 6×10⁴ cells, about 2.5×10⁴ to about 6×10⁴ cells, about 3×10⁴ to about 6×10⁴ cells, about 3.5×10⁴ to about 6×10⁴ cells, about 4×10⁴ to about 6×10⁴ cells, about 4.5×10⁴ to about 6×10⁴ cells, about 5×10⁴ to about 6×10⁴ cells, about 5.5×10⁴ to about 6×10⁴ cells, about 2×10⁴ to about 5.5×10⁴ cells, about 2.5×10⁴ to about 5.5×10⁴ cells, about 3×10⁴ to about 5.5×10⁴ cells, about 3.5×10⁴ to about 5.5×10⁴ cells, about 4×10⁴ to about 5.5×10⁴ cells, about 4.5×10⁴ to about 5.5×10⁴ cells, about 5×10⁴ to about 5.5×10⁴ cells, about 2×10⁴ to about 5×10⁴ cells, about 2.5×10⁴ to about 5×10⁴ cells, about 3×10⁴ to about 5×10⁴ cells, about 3.5×10⁴ to about 5×10⁴ cells, about 4×10⁴ to about 5×10⁴ cells, about 4.5×10⁴ to about 5×10⁴ cells, about 2×10⁴ to about 4.5×10⁴ cells, about 2.5×10⁴ to about 4.5×10⁴ cells, about 3×10⁴ to about 4.5×10⁴ cells, about 3.5×10⁴ to about 4.5×10⁴ cells, about 4×10⁴ to about 4.5×10⁴ cells, about 2×10⁴ to about 4×10⁴ cells, about 2.5×10⁴ to about 4×10⁴ cells, about 3×10⁴ to about 4×10⁴ cells, about 3.5×10⁴ to about 4×10⁴ cells, about 2×10⁴ to about 3.5×10⁴ cells, about 2.5×10⁴ to about 3.5×10⁴ cells, about 3×10⁴ to about 3.5×10⁴ cells, about 2×10⁴ to about 3×10⁴ cells, about 2.5×10⁴ to about 3×10⁴ cells, or about 2×10⁴ to about 2.5×10⁴ cells. In some embodiments, a composition can preferably include 2.5×10⁵ cells.

In some embodiments, the cell can be cultured in a medium suitable for culturing cells, such as mammalian cells, which can be human cells. The medium can be, but is not limited to fetal bovine serum (FBS) or an endothelial cell growth media.

### Method of Treating a Subject

According to the present disclosure, methods are provided for herein for treating heart disease in a subject, which can include administering to the subject a composition or a pharmaceutical composition as disclosed herein.

Also, methods are provided for herein for improving cardiac remodeling after myocardial infarction in a subject, which can include administering to the subject a composition or a pharmaceutical composition as disclosed herein.

In another aspect, methods are provided for herein for decreasing inflammation after myocardial infarction in a subject, which can include administering to the subject a composition or a pharmaceutical composition as disclosed herein.

In some embodiments, the methods described herein can have the composition or the pharmaceutical composition administered to the subject at least 10 minutes, at least 30 minutes, at least 1 hour, at least 2 hours, at least 3 hours, at least 5 hours, at least 12 hours, at least 24 hours, at least 48 hours, at least 72 hours after a myocardial infarction. The composition or the pharmaceutical composition administered to the subject between about 10 minutes to about 72 hours, about 30 minutes to about 72 hours, about 60 minutes to about 72 hours, about 2 hours to about 72 hours, about 4 hours to about 72 hours, about 6 hours to about 72 hours, about 8 hours to about 72 hours, about 10 hours to about 72 hours, about 12 hours to about 72 hours, about 14 hours to about 72 hours, about 16 hours to about 72 hours, about 18 hours to about 72 hours, about 20 hours to about 72 hours, about 22 hours to about 72 hours, about 24 hours to about 72 hours, about 36 hours to about 72 hours, about 48 hours to about 72 hours, about 10 minutes to about 48 hours, about 30 minutes to about 48 hours, about 60 minutes to about 48 hours, about 2 hours to about 48 hours, about 4 hours to about 48 hours, about 6 hours to about 48 hours, about 8 hours to about 48 hours, about 10 hours to about 48 hours, about 12 hours to about 48 hours, about 14 hours to about 48 hours, about 16 hours to about 48 hours, about 18 hours to about 48 hours, about 20 hours to about 48 hours, about 22 hours to about 48 hours, about 24 hours to about 48 hours, about 36 hours to about 48 hours, about 10 minutes to about 36 hours, about 30 minutes to about 36 hours, about 60 minutes to about 36 hours, about 2 hours to about 36 hours, about 4 hours to about 36 hours, about 6 hours to about 36 hours, about 8 hours to about 36 hours, about 10 hours to about 36 hours, about 12 hours to about 36 hours, about 14 hours to about 36 hours, about 16 hours to about 36 hours, about 18 hours to about 36 hours, about 20 hours to about 36 hours, about 22 hours to about 36 hours, about 24 hours to about 36 hours, about 10 minutes to about 24 hours, about 30 minutes to about 24 hours, about 60 minutes to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 14 hours to about 24 hours, about 16 hours to about 24 hours, about 18 hours to about 24 hours, about 20 hours to about 24 hours, about 22 hours to about 24 hours, about 10 minutes to about 22 hours, about 30 minutes to about 22 hours, about 60 minutes to about 22 hours, about 2 hours to about 22 hours, about 4 hours to about 22 hours, about 6 hours to about 22 hours, about 8 hours to about 22 hours, about 10 hours to about 22 hours, about 12 hours to about 22 hours, about 14 hours to about 22 hours, about 16 hours to about 22 hours, about 18 hours to about 22 hours, about 20 hours to about 22 hours, about 10 minutes to about 20 hours, about 30 minutes to about 20 hours, about 60 minutes to about 20 hours, about 2 hours to about 20 hours, about 4 hours to about 20 hours, about 6 hours to about 20 hours, about 8 hours to about 20 hours, about 10 hours to about 20 hours, about 12 hours to about 20 hours, about 14 hours to about 20 hours, about 16 hours to about 20 hours, about 18 hours to about 20 hours, about 10 minutes to about 18 hours, about 30 minutes to about 18 hours, about 60 minutes to about 18 hours, about 2 hours to about 18 hours, about 4 hours to about 18 hours, about 6 hours to about 18 hours, about 8 hours to about 18 hours, about 10 hours to about 18 hours, about 12 hours to about 18 hours, about 14 hours to about 18 hours, about 16 hours to about 18 hours, about 10 minutes to about 16 hours, about 30 minutes to about 16 hours, about 60 minutes to about 16 hours, about 2 hours to about 16 hours, about 4 hours to about 16 hours, about 6 hours to about 16 hours, about 8 hours to about 16 hours, about 10 hours to about 16 hours, about 12 hours to about 16 hours, about 14 hours to about 16 hours, about 10 minutes to about 14 hours, about 30 minutes to about 14 hours, about 60 minutes to about 14 hours, about 2 hours to about 14 hours, about 4 hours to about 14 hours, about 6 hours to about 14 hours, about 8 hours to about 14 hours, about 10 hours to about 14 hours, about 12 hours to about 14 hours, about 10 minutes to about 12 hours, about 30 minutes to about 12 hours, about 60 minutes to about 12 hours, about 2 hours to about 12 hours, about 4 hours to about 12 hours, about 6 hours to about 12 hours, about 8 hours to about 12 hours, about 10 hours to about 12 hours, about 10 minutes to about 10 hours, about 30 minutes to about 10 hours, about 60 minutes to about 10 hours, about 2 hours to about 10 hours, about 4 hours to about 10 hours, about 6 hours to about 10 hours, about 8 hours to about 10 hours, about 10 minutes to about 8 hours, about 30 minutes to about 8 hours, about 60 minutes to about 8 hours, about 2 hours to about 8 hours, about 4 hours to about 8 hours, about 6 hours to about 8 hours, about 10 minutes to about 6 hours, about 30 minutes to about 6 hours, about 60 minutes to about 6 hours, about 2 hours to about 6 hours, about 4 hours to about 6 hours, about 10 minutes to about 4 hours, about 30 minutes to about 4 hours, about 60 minutes to about 4 hours, about 2 hours to about 4 hours, about 10 minutes to about 2 hours, about 30 minutes to about 2 hours, about 60 minutes to about 2 hours, about 10 minutes to about 60 minutes, about 30 minutes to about 60 minutes, or about 10 minutes to about 30 minutes after a myocardial infarction.

In some embodiments, the fractional shortening can increase by at least 10%, at least 15%, at least 20%, at least 25%, at least 30%, at least 35%, at least 40%, or at least 45% compared to fractional shortening prior to administration of the composition. The fractional shortening can increase by about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 5% to about 45%, about 10% to about 45%, about 15% to about 45%, about 20% to about 45%, about 25% to about 45%, about 30% to about 45%, about 35% to about 45%, about 40% to about 45%, about 5% to about 40%, about 10% to about 40%, about 15% to about 40%, about 20% to about 40%, about 25% to about 40%, about 30% to about 40%, about 35% to about 40%, about 5% to about 35%, about 10% to about 35%, about 15% to about 35%, about 20% to about 35%, about 25% to about 35%, about 30% to about 35%, about 5% to about 30%, about 10% to about 30%, about 15% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 5% to about 15%, about 10% to about 15%, or about 5% to about 10% compared to fractional shortening prior to administration of the composition.

The fractional shortening after administration can be compared to fraction shortening prior to administration of the composition. The fractional shortening can be measured at two or more time point, including at least one measurement after the myocardial infarction and prior to administration of the composition, and at least one measurement after administration of the composition.

The at least one measurement of the fractional shortening after a myocardial infarction and prior to administration of the composition can include, but is not limited to at least 10 minutes, at least 30 minutes, at least 1 hour, at least 2 hours, at least 3 hours, at least 5 hours, at least 12 hours, at least 24 hours, at least 48 hours, at least 72 hours after the myocardial infarction. The at least one measurement of the fractional shortening after a myocardial infarction and prior to administration of the composition can include, but is not limited to at least 10 minutes, at least 30 minutes, at least 1 hour, at least 2 hours, at least 3 hours, at least 5 hours, at least 12 hours, at least 24 hours, at least 48 hours, at least 72 hours prior to the administration of the composition.

The at least one measurement of the fractional shortening after administration of the composition can include, but is not limited to between about 10 minutes to about 72 hours, about 30 minutes to about 72 hours, about 60 minutes to about 72 hours, about 2 hours to about 72 hours, about 4 hours to about 72 hours, about 6 hours to about 72 hours, about 8 hours to about 72 hours, about 10 hours to about 72 hours, about 12 hours to about 72 hours, about 14 hours to about 72 hours, about 16 hours to about 72 hours, about 18 hours to about 72 hours, about 20 hours to about 72 hours, about 22 hours to about 72 hours, about 24 hours to about 72 hours, about 36 hours to about 72 hours, about 48 hours to about 72 hours, about 10 minutes to about 48 hours, about 30 minutes to about 48 hours, about 60 minutes to about 48 hours, about 2 hours to about 48 hours, about 4 hours to about 48 hours, about 6 hours to about 48 hours, about 8 hours to about 48 hours, about 10 hours to about 48 hours, about 12 hours to about 48 hours, about 14 hours to about 48 hours, about 16 hours to about 48 hours, about 18 hours to about 48 hours, about 20 hours to about 48 hours, about 22 hours to about 48 hours, about 24 hours to about 48 hours, about 36 hours to about 48 hours, about 10 minutes to about 36 hours, about 30 minutes to about 36 hours, about 60 minutes to about 36 hours, about 2 hours to about 36 hours, about 4 hours to about 36 hours, about 6 hours to about 36 hours, about 8 hours to about 36 hours, about 10 hours to about 36 hours, about 12 hours to about 36 hours, about 14 hours to about 36 hours, about 16 hours to about 36 hours, about 18 hours to about 36 hours, about 20 hours to about 36 hours, about 22 hours to about 36 hours, about 24 hours to about 36 hours, about 10 minutes to about 24 hours, about 30 minutes to about 24 hours, about 60 minutes to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 14 hours to about 24 hours, about 16 hours to about 24 hours, about 18 hours to about 24 hours, about 20 hours to about 24 hours, about 22 hours to about 24 hours, about 10 minutes to about 22 hours, about 30 minutes to about 22 hours, about 60 minutes to about 22 hours, about 2 hours to about 22 hours, about 4 hours to about 22 hours, about 6 hours to about 22 hours, about 8 hours to about 22 hours, about 10 hours to about 22 hours, about 12 hours to about 22 hours, about 14 hours to about 22 hours, about 16 hours to about 22 hours, about 18 hours to about 22 hours, about 20 hours to about 22 hours, about 10 minutes to about 20 hours, about 30 minutes to about 20 hours, about 60 minutes to about 20 hours, about 2 hours to about 20 hours, about 4 hours to about 20 hours, about 6 hours to about 20 hours, about 8 hours to about 20 hours, about 10 hours to about 20 hours, about 12 hours to about 20 hours, about 14 hours to about 20 hours, about 16 hours to about 20 hours, about 18 hours to about 20 hours, about 10 minutes to about 18 hours, about 30 minutes to about 18 hours, about 60 minutes to about 18 hours, about 2 hours to about 18 hours, about 4 hours to about 18 hours, about 6 hours to about 18 hours, about 8 hours to about 18 hours, about 10 hours to about 18 hours, about 12 hours to about 18 hours, about 14 hours to about 18 hours, about 16 hours to about 18 hours, about 10 minutes to about 16 hours, about 30 minutes to about 16 hours, about 60 minutes to about 16 hours, about 2 hours to about 16 hours, about 4 hours to about 16 hours, about 6 hours to about 16 hours, about 8 hours to about 16 hours, about 10 hours to about 16 hours, about 12 hours to about 16 hours, about 14 hours to about 16 hours, about 10 minutes to about 14 hours, about 30 minutes to about 14 hours, about 60 minutes to about 14 hours, about 2 hours to about 14 hours, about 4 hours to about 14 hours, about 6 hours to about 14 hours, about 8 hours to about 14 hours, about 10 hours to about 14 hours, about 12 hours to about 14 hours, about 10 minutes to about 12 hours, about 30 minutes to about 12 hours, about 60 minutes to about 12 hours, about 2 hours to about 12 hours, about 4 hours to about 12 hours, about 6 hours to about 12 hours, about 8 hours to about 12 hours, about 10 hours to about 12 hours, about 10 minutes to about 10 hours, about 30 minutes to about 10 hours, about 60 minutes to about 10 hours, about 2 hours to about 10 hours, about 4 hours to about 10 hours, about 6 hours to about 10 hours, about 8 hours to about 10 hours, about 10 minutes to about 8 hours, about 30 minutes to about 8 hours, about 60 minutes to about 8 hours, about 2 hours to about 8 hours, about 4 hours to about 8 hours, about 6 hours to about 8 hours, about 10 minutes to about 6 hours, about 30 minutes to about 6 hours, about 60 minutes to about 6 hours, about 2 hours to about 6 hours, about 4 hours to about 6 hours, about 10 minutes to about 4 hours, about 30 minutes to about 4 hours, about 60 minutes to about 4 hours, about 2 hours to about 4 hours, about 10 minutes to about 2 hours, about 30 minutes to about 2 hours, about 60 minutes to about 2 hours, about 10 minutes to about 60 minutes, about 30 minutes to about 60 minutes, or about 10 minutes to about 30 minutes after administration of the composition.

In some embodiments, cells of the composition remain the heart of the subject after administration for at least about 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 34 hours, at least 36 hours, at least 38 hours, at least 40 hours, at least 42 hours, at least 44 hours, at least 46 hours, or at least 48 hours after administration of the composition. In some embodiments, about 1% to about 5% of the total number of cells in the composition can be in the heart of the subject between about 10 hours to about 24 hours after administration.

### EXAMPLES

### Example 1: Methods

hHiPC Isolation and Characterization

hHiPCs were isolated as previously described.² In brief, myocyte-depleted single cell suspensions from human ventricle epicardial biopsies were enriched for non-immune cells with the removal of CD45 cells using human anti-CD45 antibody-bound microbeads. Successful removal of immune cells was confirmed using flow cytometry.^{1,4} Cells were plated on 48-well plates at a cell density of 500 cells per cm² and formed consistent colonies with high proliferation rates, as compared to non-colony forming cells. Subsequently, hHiPCs (passage 1) were grown in Medium 199 (M199, Gibco)-Endothelial Cell Growth Medium-2 (EGM-2, Lonza); 3:1, v/v with 10% fetal bovine serum (FBS) and 1% antibiotic-anti-mycotic solution (Sigma). Human retinal endothelial cells (HRECs, Cell Systems, Kirkland, WA) were cultured in EGM-2 media at 37° C. in 5% CO₂ and used until passage 10.

In some embodiments, the heart cell can be selected from the group consisting of an epicardial cell, a pericardial cell, a cardiomyocyte, a cardiac endothelial cell, a cardiac fibroblast cell, cardiac smooth muscle cells, cardiac immune cells and an endocardial cell. In some embodiments, the isolated and clonally expanded highly proliferative cells can be between about m to about 110 μm in size across at least one axis. In some embodiments, the isolated and clonally expanded highly proliferative cells can be between about 50 μm to about 110 μm in size across at least one axis.

In some embodiments, cell division of the isolated and clonally expanded highly proliferative cells can be about 15 hours to about 90 hours. In some embodiments, cell division of the isolated and clonally expanded highly proliferative cells can be less than about 90 hours, less than about 80 hours, less than about 70 hours, less than about 60 hours, less than about 50 hours, less than about 40 hours, less than about 30 hours, less than about 20 hours, or less than about 15 hours.

In some embodiments, highly proliferative cells, which have been isolated and clonally expanded, can be defined as, but are not limited to, cardiac bipotential progenitor cells that can be differentiated into either endothelial cells or fibroblasts.

As used herein, “clonal expansion” refers to the clonal outgrowth of a population of cells derived from a common founder cell. The derivative population of cells (i.e. daughter cells), may be referred to as simply a clone. Clonal expansions can occur through artificial means (i.e. a single isolated cell in culture is allowed to grow and iteratively divide into a population of cells). Clones, by their very nature contain subclones-that is, smaller populations of cells that are clonally derived from a daughter cell of the original clonal founder. These subclones are, themselves, simply clones when viewed in reference to their specific founder cell, but are referred to as subclones when viewed relative to an even earlier founding cell. In some embodiments, subclones can be heterogeneous when comparing each daughter cell derived from the common founder cell. In another aspect, subclones can be homogeneous when comparing each daughter cell derived from the common founder cell.

### Liquid Chromatography-Tandem Mass Spectrometry (LC-MS/MS)

hHiPCs or HRECs (passage 4-6) were grown in low serum (1% FBS) for 24-hours and lysates were collected and immediately processed for LC-MS/MS, as described previously.^{2,10,11} Conditioned media (CM) were collected and concentrated using 5 kDa (Agilent) filter. Cell lysates were collected following Accutase (Thermo Fisher) detachment of cells, centrifugation, and immediately frozen at −80° C. Protein concentration was determined by BCA (Pierce). For CM, 100 μg of protein was then precipitated using 9 volumes of ethanol and frozen at −80° C. overnight. Protein was pelleted (4° C. for 20 minutes). Both CM and lysates were denatured using 8M Urea. Protein was reduced using 10 mM DTT and subsequently alkylated using 15 mM Iodoacetamide. The protein sample was then digested using 2.5 pg of trypsin (Promega) overnight at 37° C. Tryptic peptides were subsequently purified using C18 Spin columns (Pierce™), dried, and stored at −20° C. before reconstitution (2% acetonitrile (ACN), 5% Formic Acid) and LC-MS/MS analysis. For LC-MS/MS analysis with SWATH acquisition, peptide samples were analyzed on the TripleTOF 5600 System (SCIEX, Framingham, MA) following separation using a hand-fabricated nano-scale high performance C18 reverse phase liquid chromatography column (5 μm×15 cm), as previously described.¹⁰ Peptides were injected at a flow rate of 200 nL per minute with a 2-80% acetonitrile/0.1% formic acid gradient to resolve peptides.

### LC-MS/MS Data Analysis

Protein ion libraries were generated from Data dependent Acquisition (DDA) experiments and protein identifications were determined using ProteinPilot (SCIEX, version 5) software Paragon algorithm¹² with a <5% False Discovery Rate (FDR).¹³ For quantitative analysis, Variable Window SWATH with data-dependent acquisition (DDA) was conducted in triplicate and used to determine optimal Q1 isolation windows and increase specificity of detected peptide ions¹⁴. Peaks were extracted with 95% peptide confidence and 1% FDR. Quantitation was analyzed in MarkerView (Sciex) with and group comparisons were implemented using Welch's t-test¹⁵ following normalization using the most likely ratio method.¹⁶ Raw mass spectrometry data have been deposited with the ProteomeXchange Consortium via the PRIDE¹⁷ partner repository with the dataset identifier, X.

### Enrichment Analysis

Ontologic enrichment analysis of proteomic data was performed using the online GOrilla enrichment analysis and visualization tool¹⁸. GOrilla uses the standard hypergeometric model distribution for identifying enriched terms (p<0.05). Proteins were ranked from most to least significantly (p<0.05) differentially expressed.

### Single Cell RNA Sequencing Analysis

Raw data were analyzed from the dataset GSE149699¹⁹, and was obtained from the Gene Expression Omnibus (GEO) using the getGEO function from the Bioconductor package GEOquery (v2.72.0)²⁰. Files were processed using Seurat v5.1.0 R package.²¹ Briefly, cells with fewer than 200 and greater than 2500 features (genes) were filtered out and data were then normalized using the default Seurat global-scale normalization method LogNormalize (“NormalizeData” function).^{19,21,22} To correct for batch effects as experiments were performed separately, the default “IntegrateData” was used to integrate anchors using “FindIntegrationAnchors.” Subsequent workflow of normalized and integrated data included scaling and finding variable features using the standard Seurat functions. The first 14 PCA dimensions (1:14), as determined via elbow plot, were used for subsequent analysis and clustering using the default “FindNeighbors” and “FindClusters” functions. Uniform Manifold Approximation and Projections (UMAPs) were generated using “RunUMAP” function with top principal components to visualize 2D clusters. Default settings for “FindAllMarkers” were used to determine differential expression of genes of interest using the Wilcoxon rank sum test with Benjamini-Hochberg correction for multiple comparisons, as described previously¹⁹.

### Myocardial Infarction and Cell Transplantation

All studies were conducted in accordance with the Guide for the Care and Use of Laboratory Animals, as adopted and promulgated by the National Institutes of Health (Bethesda, MD). Immunocompromised NSG™ (NOD.Cg-Prkdc scid Il2rg tm1Wjl/SzJ) male and female mice (Jackson Laboratory, ME) at age of 12-16 weeks old were used to test the effect of human HiPC on cardiac function after MI. MI was induced through the ligation of left coronary artery (LCA) as described previously.²³⁻²⁵ In brief, mice were anesthetized with isoflurane, intubated and mechanically ventilated. A left thoracotomy was performed at the fourth intercostal space, and the LCA was ligated with a 7-0 suture loop. MI was confirmed by direct visualization of regional paleness of the myocardial surface below the suture and S-T segment elevation on the electrocardiogram. A bolus of hHiPC cells (passage 1, n=4 patient pooled hHiPC clones) were suspended in PBS at the cell concentration of 2×10⁶ per ml, and 125 μL of suspension was injected into 5 points (25 μL) around the peri-infarct area (2.5×10⁵ hHiPCs injected per heart). Equal volumes of cell-free PBS were injected in the same manner to a control group of animals. The chest was closed in layers, and the animals disconnected from the respirator.

### Echocardiography

Echocardiography was performed on awake, non-sedated mice using a Vevo 2100 Imaging System equipped with an 18- to 38-MHz linear-array transducer (MS400; VisualSonics, Inc., Toronto, ON, Canada). Images were acquired at a rate of 300 frames per second. Left ventricular M-mode tracings were obtained at the level of the papillary muscles using two-dimensional parasternal short axis imaging as a guide.

### Flow Cytometry

Murine hearts were perfused with PBS/heparin to remove blood, dissected to isolate ventricular tissue, which was then minced, and incubated with collagenase 11/dispase 1I/DNase I/CaCl₂ for 20 min at 37° C. Cells were treated with TruStain fcX (BioLegend) to block nonspecific binding of antibodies. The cells were subsequently incubated with the indicated antibodies for 20 minutes at 4° C. in a total volume of 100 μL, washed once with 10 volumes of cold PBS/0.5% BSA/2 mM EDTA. FITC-conjugated anti-mouse CD31 (clone: 390), APC/Cy7-CD45 (clone: 30-F11), APC-conjugated anti-human CD29 (clone: TS2/16) for cell surface staining were from BioLegend. In addition, BioLegend's antibody, including PE-conjugated anti-mouse CD11b (clone M1/70) and PE-Cy7-conjugated anti-mouse Ly6G (clone 1A8) were used to identify the granulocytic phagocytes.

Intracellular staining of human mitochondria was performed after fixation and permeabilization of cells (BD Cytofix/Cytoperm™, BD Biosciences) using biotin-conjugated anti-human Mitochondria antibody (clone: 113-1, Sigma-Aldrich) in combination with streptavidin-PE (BioLegend). Data acquisition was performed using MACSQuant Analyzer 10 (Miltenyi Biotec), and these data were analyzed with WinList 5.0 software. Nonviable cells and cell debris were excluded by using LIVE/DEAD Fixable Violet staining kit (Life Technologies). Antigen negativity was defined as having the same fluorescent intensity as the control isotype.²

### Determination of Scar Size

Infarct size was measured as described.²⁴ In brief, excised hearts were immersion-fixed in 10% buffered formalin for 24 hours and transferred to 70% ethanol, after which ventricles were cut into 3 equal parts (in length) parallel to the atrioventricular groove. From each paraffin-embedded ventricular part, 2 transverse sections (5 μm thick) were made at different levels (for a total of 6 sections). After performing Masson trichrome staining, infarct sizes were determined using the midline length measurement method.²⁶ All measurements were conducted using ImageJ software version 1.45s (National Institutes of Health) in a blinded manner.

### Statistical Analysis

Normality testing of in vivo data was calculated using Shapiro-Wilk test. The comparisons between two groups were performed using Student's t-test. SWATH data set group comparisons were analyzed using unequal variance (Welch's) t-test.^{15,26} In vivo functional data was analyzed by on-way or two-way ANOVA repeated measures with Tukey's or Sidak's multiple comparison test, respectively, and survivability by Mantel-Cox test. A p-value of less than 0.05 was considered significant.

### Example 2: Results

Unbiased proteomic characterization of hHiPCs was used to discover potential novel mechanisms of hHiPC repair. hHiPCs were observed to secrete reparative and angiogenic proteins such as CXCL6 (C-X-C motif chemokine 6), CD73 (5′-nucleotidase), and CTHRCl (Collagen triple helix repeat-containing protein 1), which are also highly transcriptionally expressed in therapeutic CDCs suggesting a potential pro-reparative role. To assess potential hHiPC pharmacologic efficacy in vivo, intramyocardial injection of hHiPCs was examined in immunocompromised adult male and female mice. After injection improved heart function and survival following experimental MI was observed. The engraftment of hHiPC was found to be associated with improved heart function after MI compared to control treatment, with a trend towards improved survivability in male mice. Together, these studies demonstrate that hHiPCs are able to promote cardiac repair, and have potential as a therapeutic cardiac cell subpopulation for treatment of myocardial injury, possibly via secretion of factors that can promote cardiac healing.

Characterization of hHiPC Protein Expression Compared to Terminally Differentiated Endothelial Cells.

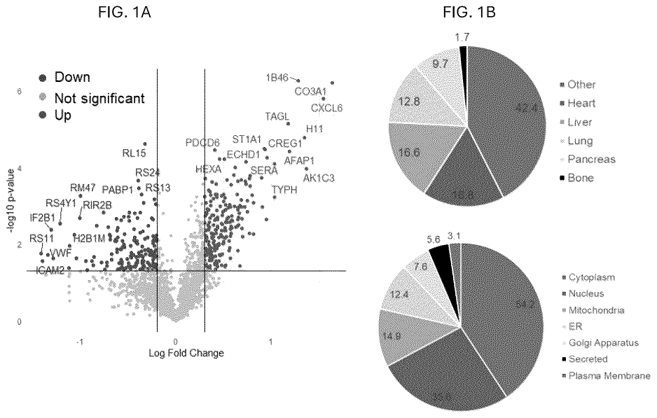

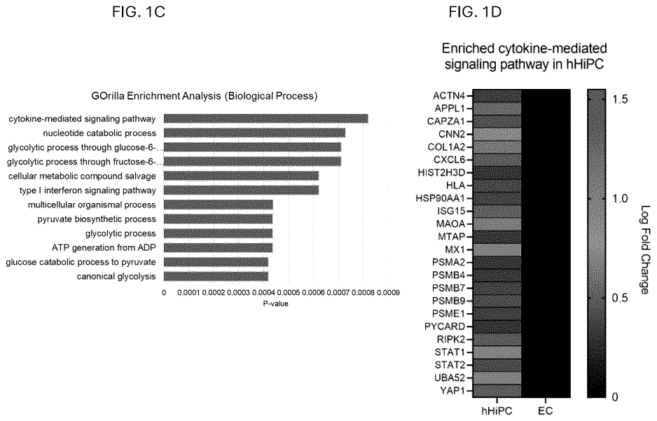

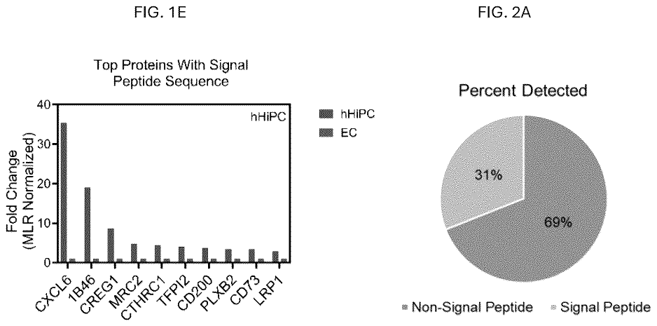

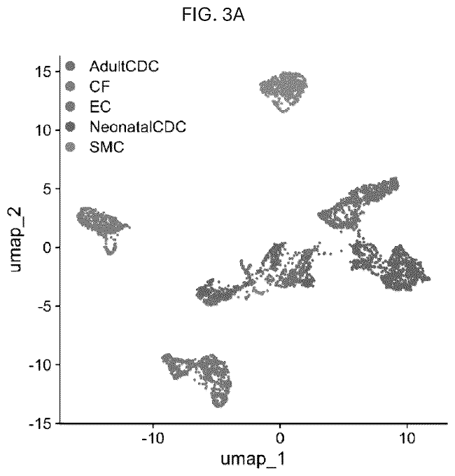

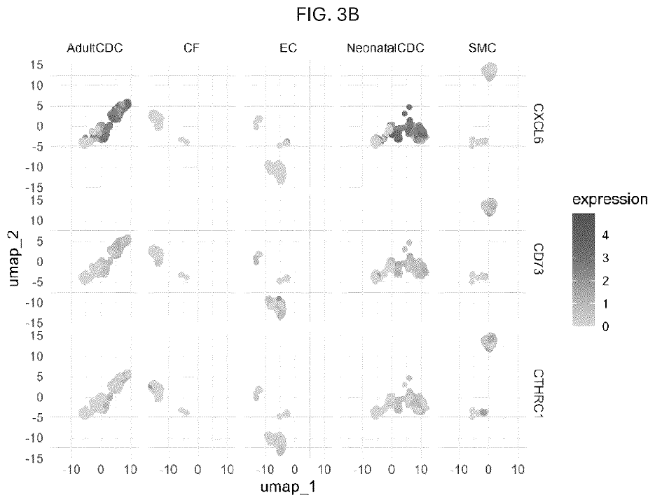

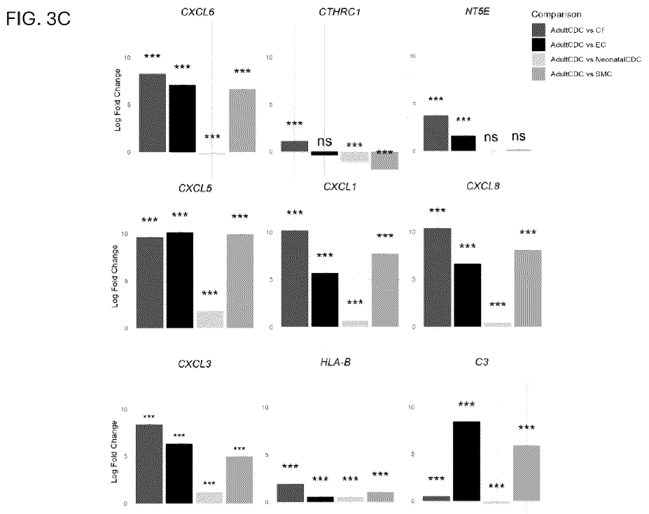

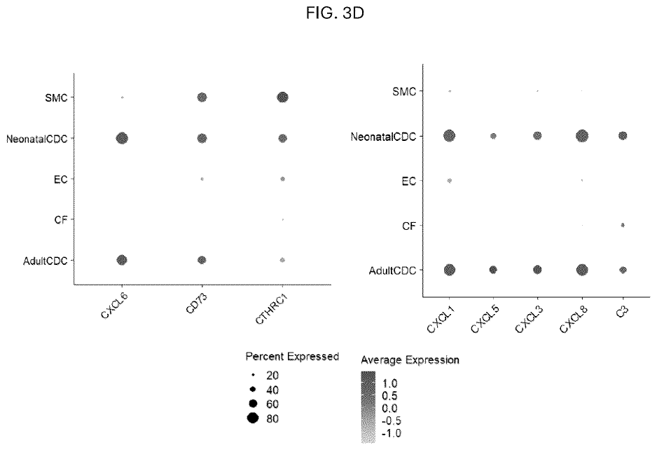

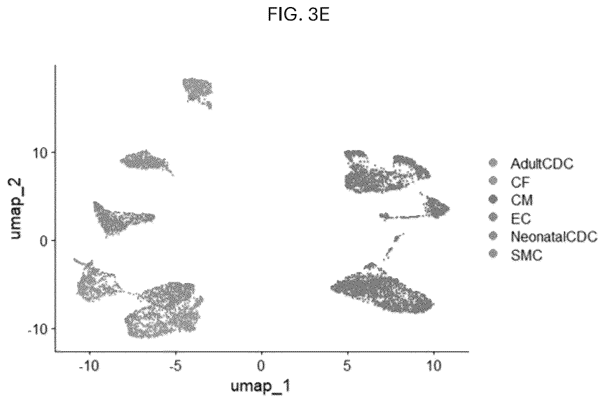

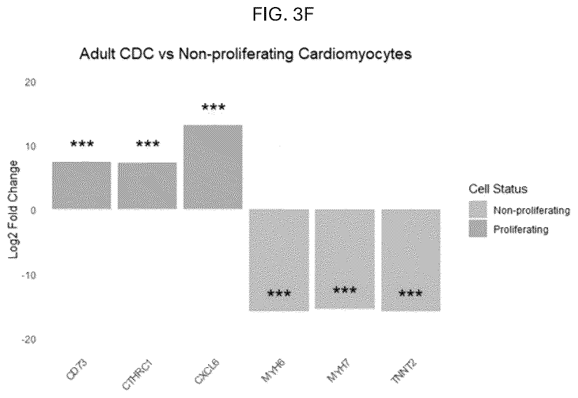

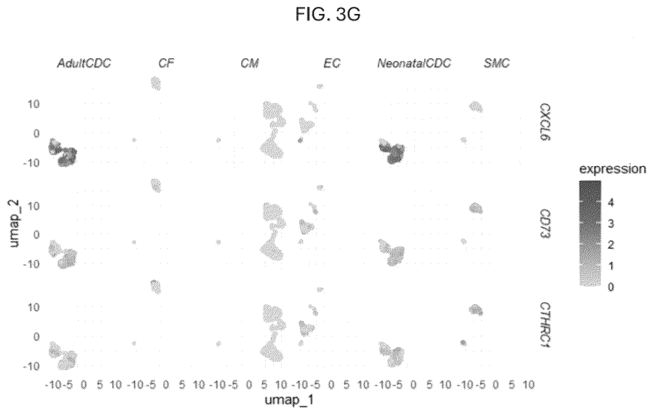

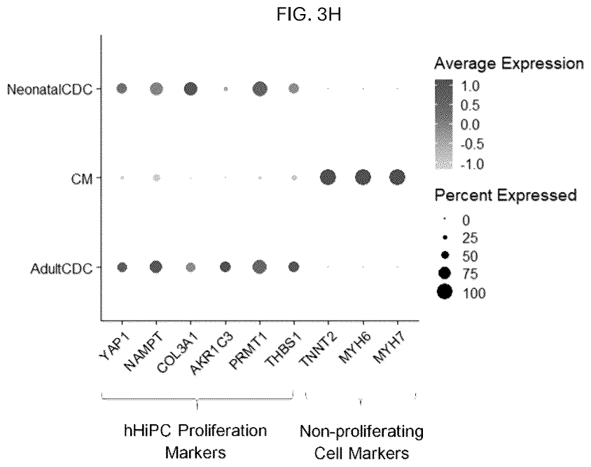

hHiPCs have been previously described as having progenitor-like properties.^{2,10} To examine whether hHiPCs express proteins that might mediate the effects of hHiPCs in vivo, proteomic analysis of 9 hHiPC clones was preformed. SWATH proteomic analysis of hHiPCs was performed to identify and quantify hHiPC-characteristic protein expression.¹⁰ 534 significantly (P<0.05) differentially expressed proteins were identified in hHiPCs, as compared with HRECs, a proangiogenic and terminally differentiated EC type. A total of 229 proteins were expressed at higher levels (P<0.05, fold change [FC]>2.0), and 153 were expressed at lower levels (P<0.05, FC<0.6) in hHiPCs than in HRECs. The top differentially expressed proteins (sorted by lowest P value) in hHiPCs are listed in Table 1. Volcano plots of the top differentially regulated proteins identified upregulated expression of proteins with therapeutic implications, such as CXCL6, COL3A1, and CREG1 and downregulated expression of EC markers, including VWF, ICAM2, EPCR, and CAV1 (FIG. 1A). No expression of cardiomyocytes markers was also observed (FIGS. 3E-3H). The top differentially expressed proteins ranked by highest and lowest fold difference in hHiPCs versus HRECs are listed in Table 2. Detection of cardiac-related proteins in hHiPCs that overlapped with the heart was based on UniProt tissue expression annotation (16.8%) and was comparable to that of liver (16.6%), lung (12.8%), pancreas (9.7%), and bone (1.7%), demonstrating that hHiPCs maintain cardiac tissue phenotypes in culture. The greatest detection of identified proteins was characterized as residing in the cytoplasm (54.2%) and nucleus (35.6%) (FIG. 1B). Gene enrichment analysis of highly expressed hHiPC proteins highlighted pathways involving cytokine signaling and glycolysis (FIG. 1C), which is consistent with previous studies investigating therapeutic cell function and fate.^{19,27,28} As cytokine signaling, such as CXCL6/CXCR activation, has been implicated in paracrine-mediated repair,^{19,28,29} cytokine-mediated signaling factors with increased expression in hHiPCs were further characterized. The analysis revealed several highly expressed paracrine mesenchymal proteins involved in regeneration and angiogenesis, such as CXCL6 and YAP1 (FIG. 1D). hHiPC proteins were assessed with an annotated signal peptide sequence (UniProt). This approach revealed the expression of secreted proteins previously identified for their involvement in cardiac repair and regeneration, which, in addition to CXCL6, included CREG1, CTHRC1, and CD73 (FIG. 1E).³⁰⁻³³ Other highly expressed signal peptide proteins, including MRC1 and MRC2, HLA-B, and CD200, have not been thoroughly investigated in the myocardium or myocardial injury. Together, these data provide an unbiased characterization of hHiPCs and identify this cell population as potentially involved in cardiac repair.

TABLE 1
Prominent secreted hHiPC proteins measured by LC-MS/MS
with SWATH acquisition (sorted by increasing p-value).

| Peak Name | p-value | Fold Change |
|---|---|---|
| HLA-B | 5.54E−07 | 19.05 |
| CO3A1 | 6.23E−07 | 43.18 |
| CXCL6 | 1.63E−06 | 35.42 |
| TAGLN | 7.25E−06 | 14.93 |
| H11 | 0.000016938 | 22.15 |
| RL15 | 0.000024553 | 0.47 |
| ST1A1 | 0.000033299 | 8.44 |
| CREG1 | 0.00003359 | 8.60 |
| PDCD6 | 0.000035974 | 2.55 |
| AFAP1 | 0.000038117 | 15.57 |
| SERA | 0.000056611 | 9.09 |
| HEXA | 0.000061362 | 2.86 |
| ECHD1 | 0.000061961 | 3.22 |
| MYO5A | 0.000073286 | 5.45 |
| CHM2B | 0.000081286 | 10.73 |
| CO1A2 | 0.0001 | 4.20 |
| AK1C3 | 0.00011 | 23.46 |
| EWS | 0.00012 | 1.66 |
| MMSA | 0.00016 | 4.05 |

TABLE 2
Most upregulated (bold) and downregulated (underlined) hHiPC
proteins measured by LC-MS/MS with SWATH acquisition.

| Peak Name | p-value | Fold Change |
|---|---|---|
| CO3A1 | 6.23E−07 | 43.18 |
| CXCL6 | 1.63E−06 | 35.42 |
| AK1C3 | 0.00011 | 23.46 |
| H11 | 0.000016938 | 22.15 |
| HLA-B | 5.54E−07 | 19.05 |
| AFAP1 | 0.000038117 | 15.57 |
| TAGLN | 7.25E−06 | 14.93 |
| TYPH | 0.0006 | 10.74 |
| CHM2B | 0.000081286 | 10.73 |
| SERA | 0.000056611 | 9.09 |
| RS11 | 0.01761 | 0.03 |
| ICAM2 | 0.02811 | 0.04 |
| R39L5 | 0.01925 | 0.045 |
| IF2B1 | 0.00439 | 0.04 |
| EPCR | 0.02368 | 0.05 |
| RS4Y1 | 0.00299 | 0.06 |
| SYDC | 0.04326 | 0.07 |
| VWF | 0.01118 | 0.07 |
| H2B1M | 0.00587 | 0.08 |
| ACTG | 0.02353 | 0.09 |

hHiPC Secretome Identifies Cardiac Repair Paracrine Factors.

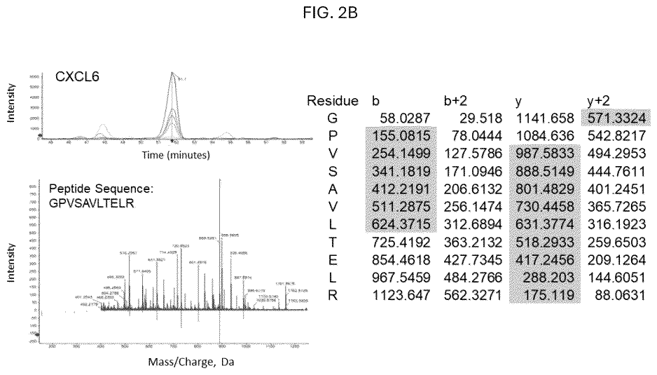

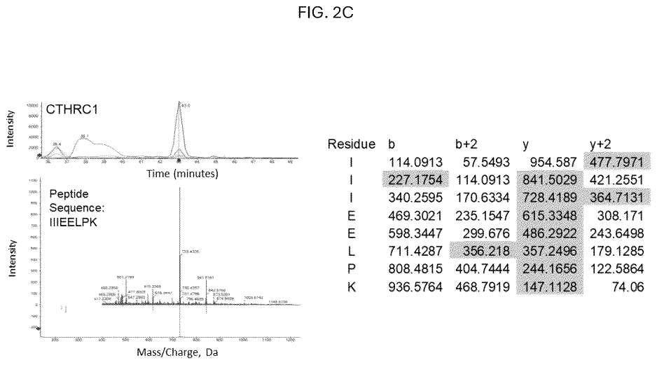

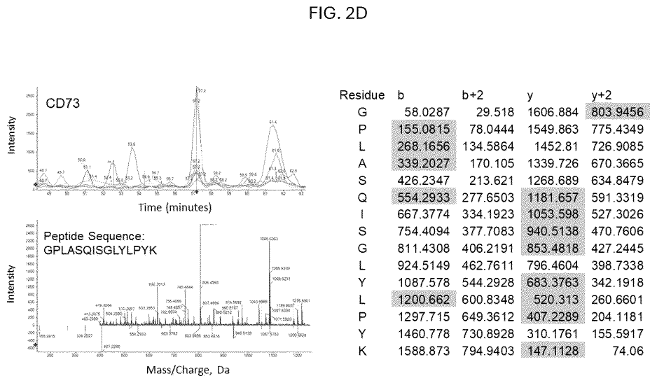

LC-MS/MS was used to analyze hHiPC CM and to identify highly expressed proteins found in the secretome. A total of 552 proteins were detected with high confidence (>95%) by DIA and a table of the top secreted proteins identified in hHiPCs is outlined in Table 3. Among these detected proteins, 172 proteins were identified to have a signal peptide sequence (FIG. 2A). Confirming identified targets of interest with a signal peptide sequence, spectra for CD73, CTHRC1, and CXCL6 peptides in the hHiPC secretome were identified (FIGS. 2B-D). These observations support the hypothesis that hHiPCs are a rich source of secreted proteins, including CD73, CTHRC1, and CXCL6, and may suggest that hHiPCs harbor the innate ability to promote cardiac repair.

TABLE 3
List of the top 25 secreted proteins identified by LC-MS/MS
DIA (sorted by the Paragon algorithm unused function)¹².

| | UNUSED | | PEPTIDES (95% |
|---|---|---|---|
| N | (>1.3) | ACCESSION | CONFIDENCE) |
| 1 | 253.45 | P02751\|FINC_HUMAN | 238 |
| 2 | 215.32 | P98160\|PGBM_HUMAN | 110 |
| 3 | 165.92 | P01024\|CO3_HUMAN | 101 |
| 4 | 163.3 | P12111\|CO6A3_HUMAN | 82 |
| 5 | 155.4 | P35555\|FBN1_HUMAN | 88 |
| 6 | 133.83 | P08123\|CO1A2_HUMAN | 113 |
| 7 | 130.71 | P07996\|TSP1_HUMAN | 102 |
| 8 | 80.84 | P02452\|CO1A1_HUMAN | 48 |
| 9 | 79.59 | P08603\|CFAH_HUMAN | 44 |
| 10 | 78.61 | Q15063\|POSTN_HUMAN | 57 |
| 11 | 75.68 | P08253\|MMP2_HUMAN | 41 |
| 12 | 65.86 | P21333\|FLNA_HUMAN | 37 |
| 13 | 62.86 | O43707\|ACTN4_HUMAN | 36 |
| 14 | 62.2 | P35442\|TSP2_HUMAN | 40 |
| 15 | 62.06 | O00468\|AGRIN_HUMAN | 31 |
| 16 | 61.54 | Q15582\|BGH3_HUMAN | 50 |
| 17 | 60.89 | Q14767\|LTBP2_HUMAN | 34 |
| 18 | 58.99 | Q92626\|PXDN_HUMAN | 32 |
| 19 | 57.36 | P12109\|CO6A1_HUMAN | 33 |
| 20 | 56.34 | Q13219\|PAPP1_HUMAN | 33 |
| 21 | 52.07 | P05121\|PAI1_HUMAN | 63 |
| 22 | 51.33 | P00736\|C1R_HUMAN | 31 |
| 23 | 50.91 | P14618\|KPYM_HUMAN | 28 |
| 24 | 50.75 | P08670\|VIME_HUMAN | 42 |
| 25 | 48.27 | P11142\|HSP7C_HUMAN | 25 |

Comparative Analysis of hHiPC with Adult and Neonatal CDC.

Since hHiPCs express potentially interesting paracrine proteins involved in improving cardiac repair publicly available single-cell datasets from cardiac surgery patients was utilized (GSE149699) to further elucidate whether factors secreted by hHiPC (CXCL6, CTHRC1, CD73) are relevant to adult and neonatal cardiospheres derived cells (CDCs), a cell-type currently being tested in clinical trials.¹⁹ To compare across major cell types of the adult heart, UMAPs of single-cell data were generated for adult cardiospheres derived cells (Adult CDC), cardiac fibroblasts (CF), endothelial (EC), smooth muscle cells (SMC), and neonatal cardiospheres derived cells (Neonatal CDC) (FIG. 3A). Gene expression UMAPs generated for the prioritized secreted factors CXCL6, CTHRC1, and CD73 (FIG. 3B) showed high expression in adult and neonatal CDC (purple), with less expression in SMC (light purple), and very low expression in CF and EC (light purple/grey). Comparisons were then performed to determine statistical significance for CXCL6, CTHRC1, and NT5E (CD73) expression across these cell populations. Transcriptional expression of CXCL6 was statistically higher in adult CDC compared to EC, SMC, and CF, but not neonatal CDC, which are known for their strong regenerative potential and highlight CXCL6 as potentially highly characteristic of regenerative effects (FIG. 3C)³⁴ CTHRC1 expression was higher in adult CDC compared to CF, but lower when compared to SMC and neonatal CDC. There are no differences in CTHRC1 between adult CDC and EC suggesting CTHRC1 as most characteristic of SMC and neonatal CDC, at least on the mRNA level. Interestingly, with characteristic expression of CTHRC1 in neonatal CDC, this may suggest a significant role in regenerative and rejuvenation effects.³⁵ NT5E is higher in adult CDC compared to CF and EC, but no differences were found between SMC and neonatal CDC supporting the notion that NT5E is characteristic of adult and neonatal CDC as well as SMC (FIG. 3C). Using a publicly available single-cell dataset to assess control nonproliferating adult cardiomyocytes (GSE109816), CXCL6, CD73, and CTHRC1 were found to be highly expressed in adult CDCs compared with adult cardiomyocytes (FIGS. 3E and 3F) and showed very low to no expression in adult cardiomyocytes (FIG. 3G). Expression of mature cardiomyocyte markers (e.g., MYH6, MYH7, TNNT2) was also decreased in adult CDCs (FIG. 3F). Further, pro-proliferative protein factors highly expressed in hHiPCs, such as YAP1, NAMPT, COL3A1, AKR1C3, PRMT1, and THBS1, were not expressed in nonproliferating cardiomyocytes of the adult human heart (FIG. 311), highlighting the pro-proliferative and differentiation potential of hHiPCs. Other secreted proteins found in hHiPC, such as CXCL5, CXCL1, CXCL3, CXCL8, and CO3, showed high mRNA expression in adult and neonatal CDC compared to all other cardiac cell types (FIG. 3C).

Finally, dot plots were generated from the single-cell data and found >40% cell expression of CXCL6, NT5E, and CTHRC1 in both adult and neonatal CDCs (FIG. 3D). Other interesting targets found in the hHiPC secretome are also analyzed and highly expressed in adult and neonatal CDC clusters including CXCL1, CXCL5, CXCL3, CXCL8, and CO3 (FIGS. 3C-3D). These data support the hypothesis that hHiPCs harbor secreted factors characteristic of therapeutic cells and provide the basis for future mechanistic studies.

Transplanted hHiPC Survive the Acute Phase of MI

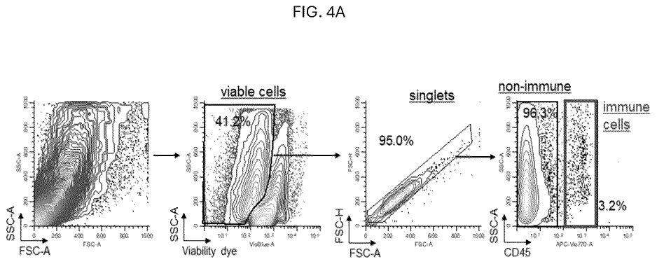

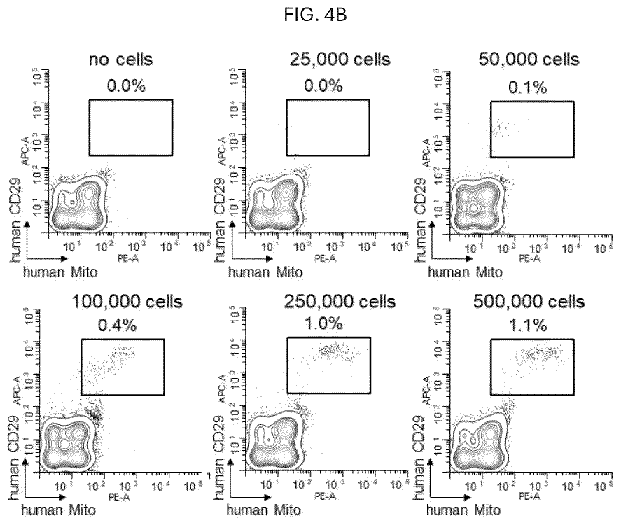

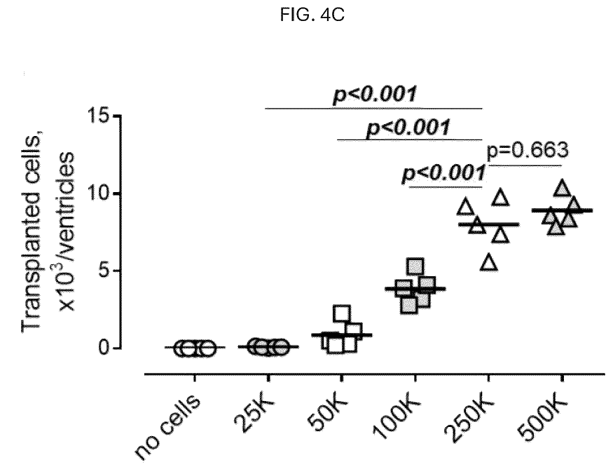

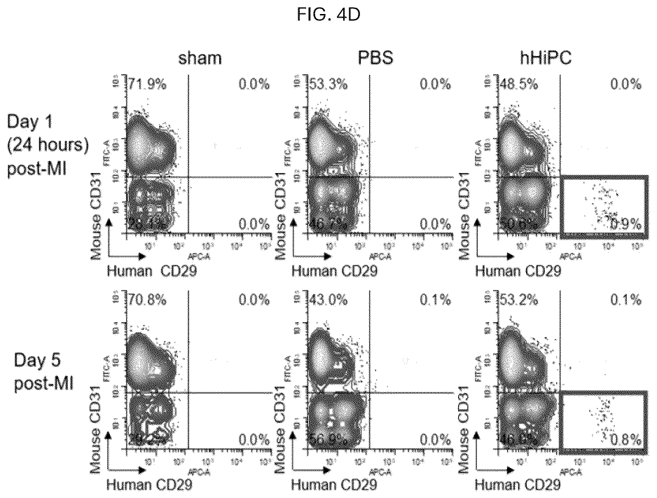

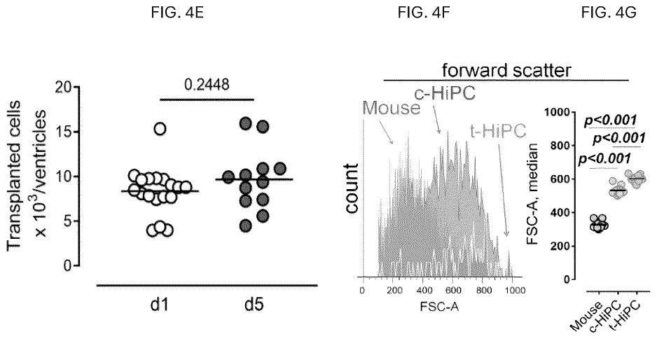

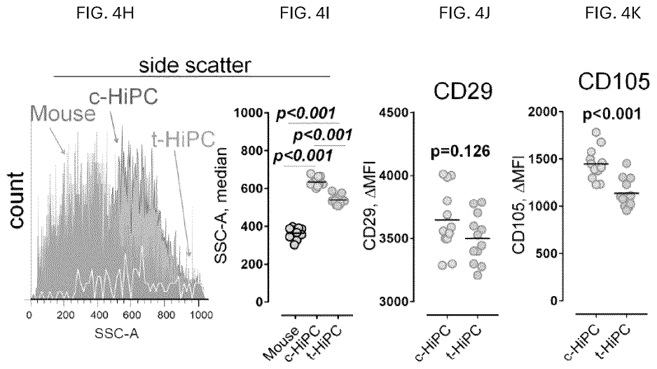

A key advantage of stem cell therapy over other approaches is cell ability to engraft into patient cardiac tissue, supporting prolonged therapeutic efficacy over-time compared to repeated drug treatments. To determine the efficiency of hHiPC transplantation, first, the effect of different concentrations of hHiPC cells injected into the uninjured heart was tested. In addition to blocking of Fcγ receptors, dead cells, cell debris, cell aggregates, and CD45 immune cells were excluded from the analysis to avoid any potential non-specific binding of anti-human antibodies (FIG. 4A). Human HiPC cells were identified using antibodies against two human antigens, cell surface CD29/β1 integrin and intracellular human mitochondria, clone 113-1. As shown in FIG. 4B, this approach allowed the identification of a distinct subset of cells after the transplantation that are absent in mouse hearts not injected with human cells. Both antibodies demonstrated high specificity. However, CD29 exhibited higher expression levels, and did not overlap with mouse cells, as specifically shown by the lower number of engrafted cells (after the injection of 0.25×10⁵, 0.5×10⁵, and 1.0×10⁵ cells), compared to human Mitochondria antibody (FIG. 4B). Injection of 2.5×10⁵ and 5.0×10⁵ hHiPC cells resulted in the highest number of transplanted cells 24 hours after the injection (FIG. 4C). The cell concentration of 2.5×10⁵ hHiPC per heart was chosen for the next experiments because it demonstrated the similar number of transplanted cells to the injection of 5.0×10⁵ hHiPC. This dosage (<500,000 cells) has also been shown in previous CDC dosage studies in mice to improve therapeutic outcomes.³⁶

To understand the fate of transplanted hHiPCs in the injured heart, flow cytometry analysis was performed for cells isolated from the infarcted heart before and after the acute phase of MI where there is the highest occurrence of cell death.^{37,38} The analysis of cells expressing human CD29 was used to determine the total number of viable transplanted hHiPC present in the left ventricle at 24 hours (peak inflammation) and 5 days (resolution phase) after MI (FIG. 4D). 3.5% (~8.7×10³ cells) of the total injected hHiPC were present in the left ventricles at 24 hours post-MI (FIG. 4E). Moreover, the total number of viable hHiPC did not change following the acute phase of MI. The small but stable number of hHiPC present after MI, in association with a late effect on remodeling and cardiac function, support a potential functional role of hHiPC-derived paracrine factors as regulators of myocardial repair.

Intramyocardial Injection of hHiPCs Following Myocardial Infarction in NSG Mice Improves Heart Function.

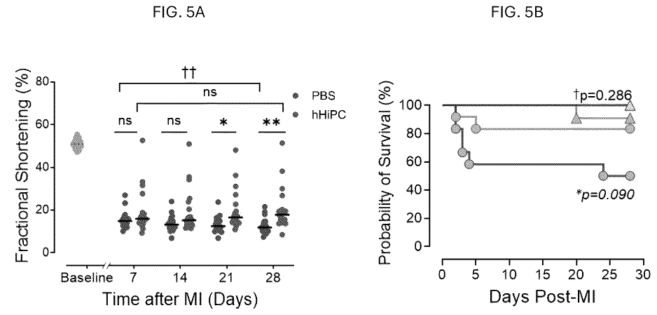

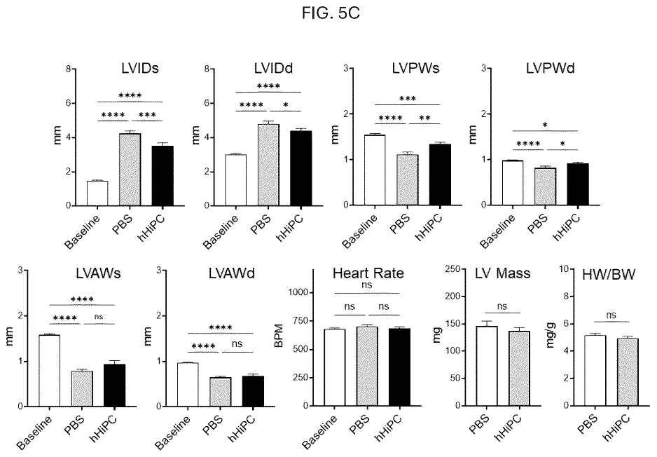

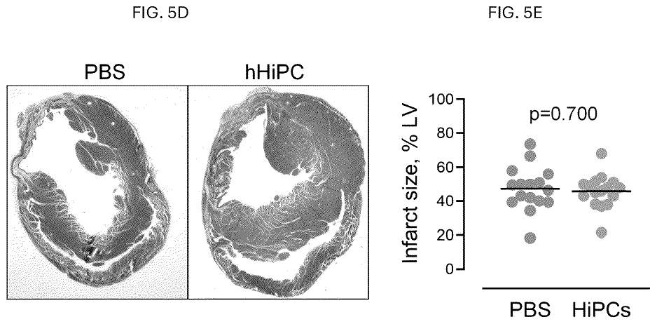

Following LCA to induce experimental MI, hHiPC cells were injected into the infarct border zone and measured outcomes over 28-days to assess changes in ventricular function and remodeling. Using non-invasive echocardiographic analysis, the treatment with hHiPC injection was determined to improved cardiac function as assessed by fractional shortening percentage 21- and 28-days post-MI, as compared to PBS controls (FIG. 5A). Female mice had higher survivability (90-100%) following MI irrespective of hHiPC or PBS treatment, compared to male NSG mice. Interestingly, in male mice, we saw a trend toward improved survivability after hHiPC injection (30% improved survival) compared to PBS treated controls, although this observation did not achieve statistical significance (FIG. 5B). Additional analysis of heart function showed improvement in LV contractility and remodeling in hHiPC-treated mice compared to controls (FIG. 5C) supporting the hypothesis that engrafted hHiPCs are involved in mediating cardiac repair. Together, these findings support a novel cell population that is easily expandable from patient cardiac tissue and has beneficial reparative effects in vivo.

Clonal Expansion of hHiPCs

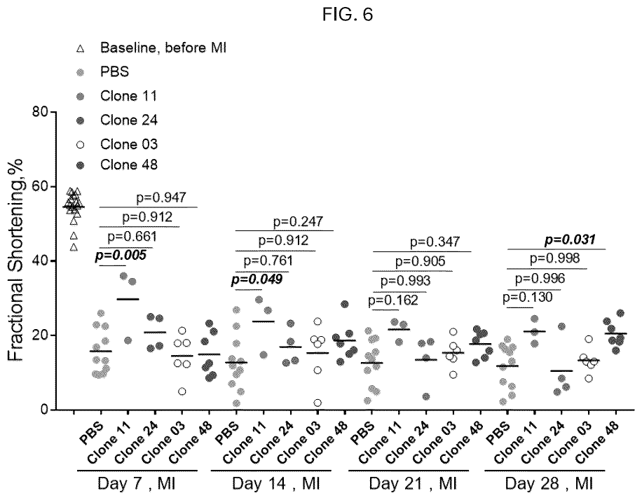

Experimental myocardial infarction was induced in 15-16 weeks-old mice. A total of 2.5×10⁵ HiPC cells per heart (four clones, each from a different individual) were injected into the peri-infarct area approximately 10 minutes later after MI. Cells for injections were prepared in parallel and grown in similar conditions for two passages (P2). The differences in the injected animals are due to the difference in cell availability (cells obtained from different clones are characterized by different growth dynamics). Number of injected animals: PBS, n=11; Clone 11, n=3; Clone 24, n=4; Clone 03, n=6; Clone 48, n=7. P values were calculated using RM two-way ANOVA, Tukey's multiple post-test. (FIG. 6)

Intramyocardial Transplantation of hHiPCs Promotes Accumulation of Granulocytic Phagocytes after Myocardial Ischemic Injury.

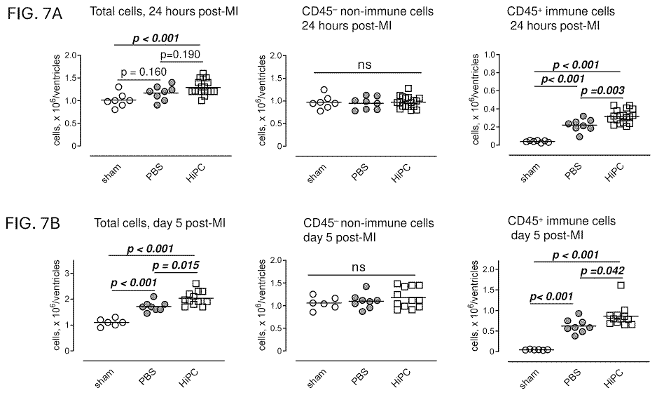

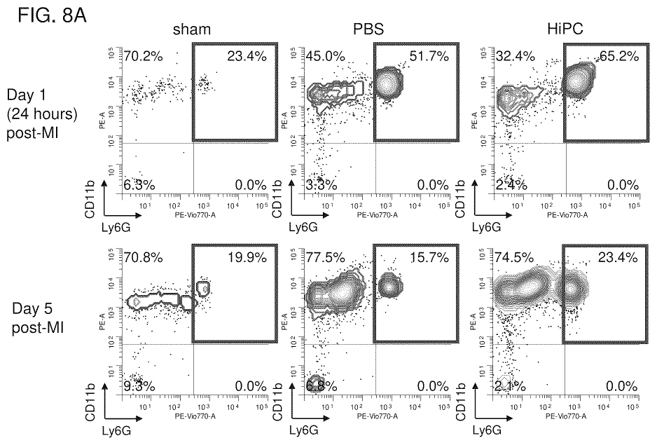

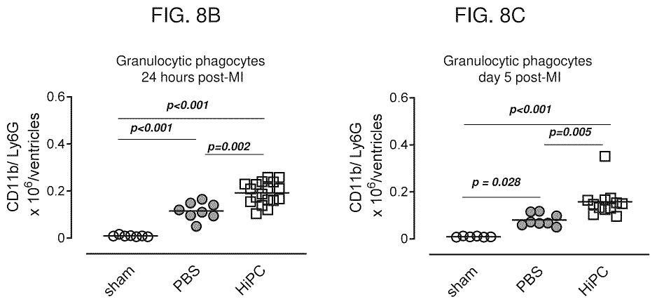

As shown in FIGS. 7A and 7B, the injection of hHiPC resulted in accumulation of CD45 positive immune cells compared to control injection of PBS as early as 24 hr (FIG. 7A) and continued until day 5 (FIG. 7B) after induction of myocardial infarction in mice. To determine subpopulation of immune cells, markers of myeloid cells were analyzed, and specifically granulocytic phagocytes, including CD11b and Ly6G (FIG. 8A). The population of granulocytic phagocytes was significantly higher in the myocardium of mice injected with hHiPC compared to control animals (PBS) on 24 hr (FIG. 8B) and day 5 (FIG. 8C) after the MI.

CTHRC1 Promotes Healing after the Myocardial Infarction.

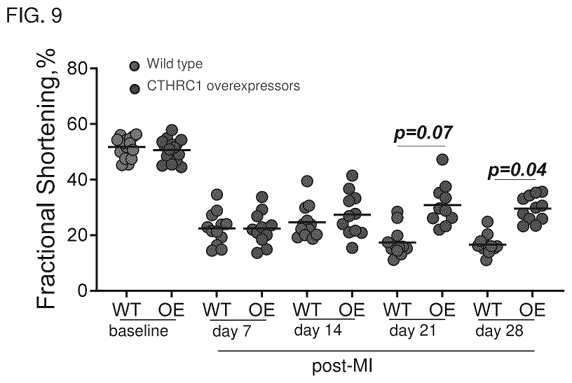

The data demonstrated that hHiPC cells produce high level of CTHRC1 (FIG. 2C). To determine the effect of CTHRC1 in myocardial repair, analysis of cardiac function in mice, which were genetically modified to produce high level of CTHRC1 (overexpresssors), was performed. As shown in FIG. 9, the cardiac function was significantly improved in mice with overexpression of CTHRC1 compared to wild type control animals, indicating that CTHRC1 promotes healing after the MI.

### Discussion

The results demonstrate that intramyocardial treatment with hHiPCs following MI improved heart function and prevented progressive cardiac remodeling in mice. No study has investigated this cell population's role in myocardial repair in vivo. Several paracrine factors characteristically enriched in hHiPC were identified, including CD73, CXCL6, and CTHRC1. The identified paracrine factors may be involved in the beneficial response. These data and others provide a potential mechanistic foundation to better understand how hHiPC contribute to myocardial infarction and repair. Further, the data highlights the hHiPC secretome for further study in basic myocardial biology as well as potential therapy in heart failure.

Cell therapies have pro-angiogenic activity that are thought to contribute to cardiac repair, both through direct differentiation into endothelial cells or through paracrine stimulation of endogenous cells.^{6,25,28,39} hHiPCs are an expandable cell population from the adult heart tissue that harbor intrinsic angiogenic properties similar to cardiospheres derived cells (CDCs), which have been used in several clinical trials.^{2,40-42} hHiPCs and CDCs are similarly characterized by cell surface expression of CD105, CD90, CD29, and newly identified marker, CXCL6.^{2,10,19,26,29} Importantly, hHiPCs do not express other characteristic markers of CDC such as c-Kit (CD117) and CD34, identifying hHiPCs as a specific sub-population of cardiac progenitor-like cells.² Additionally, hHiPCs were found to express ALK1 (activin A receptor like type 1), a co-receptor for CD105, and respond to BMP9 treatment to regulate angiogenesis and CD105, SOST, ISLR, and IGFBP3 expression.¹⁰ hHiPCs injected into the peri-infarct area of mice with experimental MI improved heart function with a reversal in the trend toward heart failure post-MI, and a trend towards increased post-infarct survival. These findings support the concept that hHiPCs are an easily expandable sub-population of cells in the human adult heart that harbor the intrinsic ability to promote repair.

In the present study of the hHiPC secretome, key factors were previously reported as myocardial repair proteins involved in processes such as angiogenesis, regeneration, and inflammation. Among them is CXCL6, a secreted cytokine of the CXC chemokine family, which is known to play a role in neutrophil degranulation and signals through binding of CXCR1 and CXCR2.^{44,45} CXCR1 and CXCR2 play prominent roles in CVD pathophysiology and regulate many cellular processes in the heart including inflammation, cell migration, angiogenesis, and fibrosis.⁴⁶ Recently, CXCL6 was identified as a functional paracrine factor in the human cardiac progenitor cell secretome, and has a pro-angiogenic role during cardio-toxic injury in mice.²⁹ However, no studies have yet investigated the effect of global or conditional disruption of CXCL6 expression.

Other secreted proteins expressed in hHiPC and involved in cardiovascular maintenance include CD73 and CTHRC1. CD73, or ecto-5′-nucleotidase, is a mesenchymal stem cell marker and has an anti-inflammatory role in the heart through production of adenosine.^{31,47,48} Recently, CD73 was identified as a prominent factor in cell therapy-induced repair in a swine model of MI, and CD73 expressing mesenchymal stem cells improved myocardial recovery through angiogenesis in a rat model of MI.^{32,49} Adenosine generated by CD73 and acting through adenosine receptors increased production of vascular endothelial growth factor in mouse cardiac stromal cells.⁵⁰ Further, CD73 has also been shown to enhance homing of circulating endothelial progenitors to the heart.⁵¹ Therefore, secretion of CD73 from hHiPC may promote repair, in part, through induction of immunosuppression and angiogenesis.

Another interesting paracrine protein enriched in hHiPC is CTHRC1, or Collagen triple helix repeat containing 1. CTHRC1 is predominantly expressed in cardiac fibroblasts to regulate repair.⁵² CTHRC1 promotes endothelial function and glycolysis in vitro.³³ CTHRC1 is increased in the circulation of patients following MI, and a disruption of Cthrc1 expression worsens cardiac outcomes in mice following ischemic injury, potentially through increased production of inflammatory markers MMP2 and MMP9.⁵³ Therefore, hHiPCs may regulate cardiac healing in vivo through CTHRC1 secretion.

Other potentially interesting proteins in hHiPC and CDC include, but are not limited to, CXCL5, CXCL1, CXCL3, CXCL8, AGRIN, and CO3. Secreted factors CXCL5, CXCL1, CXCL3, and CXCL8 are chemokines known to be involved in neutrophil recruitment and modulation of the immune response through CXCR receptor signaling which can have both inflammatory and angiogenic effects in the heart.^{54,55} CO3, complement component 3, is a secreted protein involved in cardiac regeneration and stem cell mediated-repair and recruitment.^{56,57} Further, C3 knockout mice show reduced heart function, decreased capillary density, increased apoptosis, and increased scar size following MI suggesting the important role of C3 in tissue repair.⁵⁶ Other compelling paracrine proteins among the hHiPC list include AGRIN, a secreted protein involved in cardiomyocyte regeneration and repair in murine and porcine models of MI.^{58,59} With high expression of these paracrine factors in both hHiPC and CDC, this may highlight additional targets for investigation in hHiPC-mediated secretome repair where a cocktail of specific proteins may be needed for enhanced therapeutic benefit in cell 10,60,61 therapy^{10,60,61}.

Secreted proteins can initiate processes that play an important role in cell therapy and cardiac repair mechanisms.²⁷ The studies provided herein are the first to find that a population of highly proliferative cells that reside in the adult human myocardium can improve heart function in mice following MI.

### REFERENCES

ADDIN EN.REFLIST 1. Nguyen P K, Rhee J-W, Wu J C. Adult stem cell therapy and heart failure, 2000 to 2016: a systematic review. JAMA cardiology. 2016; 1:831-841.
2. Ryzhov S, Robich M P, Roberts D J, Favreau-Lessard A J, Peterson S M, Jachimowicz E, Rath R, Vary C P, Quinn R, Kramer R S. ErbB2 promotes endothelial phenotype of human left ventricular epicardial highly proliferative cells (eHiPC). Journal of molecular and cellular cardiology. 2018; 115:39-50.
3. Na T, Liu J, Zhang K, Ding M, Yuan B Z. The notch signaling regulates CD105 expression, osteogenic differentiation and immunomodulation of human umbilical cord mesenchymal stem cells. PLoS One. 2015; 10:e0118168. doi: 10.1371/journal.pone.0118168
4. Mark P, Kleinsorge M, Gaebel R, Lux C A, Toelk A, Pittermann E, David R, Steinhoff G, Ma N. Human Mesenchymal Stem Cells Display Reduced Expression of CD105 after Culture in Serum-Free Medium. Stem cells international. 2013; 2013:698076. doi: 10.1155/2013/698076
5. Arthur H M, Ure J, Smith A J, Renforth G, Wilson D I, Torsney E, Charlton R, Parums D V, Jowett T, Marchuk D A, et al. Endoglin, an ancillary TGFbeta receptor, is required for extraembryonic angiogenesis and plays a key role in heart development. Dev Biol. 2000; 217:42-53.
6. Redgrave R E, Tual-Chalot S, Davison B J, Singh E, Hall D, Amirrasouli M M, Gilchrist D, Medvinsky A, Arthur H M. Cardiosphere-derived cells require endoglin for paracrine-mediated angiogenesis. Stem cell reports. 2017; 8:1287-1298.
7. Mishra R, Vijayan K, Colletti E J, Harrington D A, Matthiesen T S, Simpson D, Goh S K, Walker B L, Almeida-Porada G, Wang D. Characterization and functionality of cardiac progenitor cells in congenital heart patients. Circulation. 2011; 123:364-373.
8. Hou M, Yang K-m, Zhang H, Zhu W-Q, Duan F-j, Wang H, Song Y-h, Wei Y-j, Hu S-s. Transplantation of mesenchymal stem cells from human bone marrow improves damaged heart function in rats. International journal of cardiology. 2007; 115:220-228.
9. Marino F, Scalise M, Cianflone E, Mancuso T, Aquila I, Agosti V, Torella M, Paolino D, Mollace V, Nadal-Ginard B. Role of c-kit in myocardial regeneration and aging. Frontiers in endocrinology. 2019; 10:371.
10. Moore M, Ryzhov S, Sawyer D B, Gartner C, Vary C P. ALK1 Signaling in Human Cardiac Progenitor Cells Promotes a Pro-angiogenic Secretome. Journal of cellular signaling. 2024; 5:122-142.
11. Young K, Conley B, Romero D, Tweedie E, O'Neill C, Pinz I, Brogan L, Lindner V, Liaw L, Vary C P. BMP9 regulates endoglin-dependent chemokine responses in endothelial cells. Blood, The Journal of the American Society of Hematology. 2012; 120:4263-4273.
12. Shilov I V, Seymour S L, Patel A A, Loboda A, Tang W H, Keating S P, Hunter C L, Nuwaysir L M, Schaeffer D A. The Paragon Algorithm, a next generation search engine that uses sequence temperature values and feature probabilities to identify peptides from tandem mass spectra. Mol Cell Proteomics. 2007; 6:1638-1655. doi: T600050-MCP200 [pii]10.1074/mcp.T600050-MCP200
13. Tang W H, Shilov I V, Seymour S L. Nonlinear fitting method for determining local false discovery rates from decoy database searches. J Proteome Res. 2008; 7:3661-3667. doi: 10.1021/pr070492f
14. Schubert O T, Gillet L C, Collins B C, Navarro P, Rosenberger G, Wolski W E, Lam H, Amodei D, Mallick P, MacLean B, et al. Building high-quality assay libraries for targeted analysis of SWATH M S data. Nat Protoc. 2015; 10:426-441. doi: 10.1038/nprot.2015.015
15. Ivosev G, Burton L, Bonner R. Dimensionality reduction and visualization in principal component analysis. Analytical chemistry. 2008; 80:4933-4944. doi: 10.1021/ac800110w
16. Lambert J P, Ivosev G, Couzens A L, Larsen B, Taipale M, Lin Z Y, Zhong Q, Lindquist S, Vidal M, Aebersold R, et al. Mapping differential interactomes by affinity purification coupled with data-independent mass spectrometry acquisition. Nat Methods. 2013; 10:1239-1245. doi: 10.1038/nmeth.2702
17. Perez-Riverol Y, Bai J, Bandla C, Garcia-Seisdedos D, Hewapathirana S, Kamatchinathan S, Kundu D J, Prakash A, Frericks-Zipper A, Eisenacher M, et al. The PRIDE database resources in 2022: a hub for mass spectrometry-based proteomics evidences. Nucleic Acids Res. 2022; 50:D543-D552. doi: 10.1093/nar/gkab1038
18. Eden E, Navon R, Steinfeld I, Lipson D, Yakhini Z. GOrilla: a tool for discovery and visualization of enriched GO terms in ranked gene lists. BMC bioinformatics. 2009; 10:1-7.
19. Kogan P-S, Wirth F, Tomar A, Darr J, Teperino R, Lahm H, Drelβen M, Puluca N, Zhang Z, Neb I. Uncovering the molecular identity of cardiosphere-derived cells (CDCs) by single-cell RNA sequencing. Basic Research in Cardiology. 2022; 117:11.
20. Davis S, Meltzer P S. GEOquery: a bridge between the Gene Expression Omnibus (GEO) and BioConductor. Bioinformatics. 2007; 23:1846-1847.
21. Satija R, Farrell J A, Gennert D, Schier A F, Regev A. Spatial reconstruction of single-cell gene expression data. Nature biotechnology. 2015; 33:495-502.
22. Willie E. Robust methods for inferring cluster structure in Single Cell RNA Sequencing data. In: University of British Columbia; 2020.
23. Asson-Batres M A, Ryzhov S, Tikhomirov O, Duarte C W, Congdon C B, Lessard C R, McFarland S, Rochette-Egly C, Tran T-L, Galindo C L. Effects of vitamin A deficiency in the postnatal mouse heart: role of hepatic retinoid stores. American Journal of Physiology-Heart and Circulatory Physiology. 2016; 310:H1773-H1789.
24. Ryzhov S, Zhang Q, Biaggioni I, Feoktistov I. Adenosine A2B receptors on cardiac stem cell antigen (Sca)-1-positive stromal cells play a protective role in myocardial infarction. The American journal of pathology. 2013; 183:665-672.
25. Ryzhov S, Goldstein A E, Novitskiy S V, Blackburn M R, Biaggioni I, Feoktistov I. Role of A2B adenosine receptors in regulation of paracrine functions of stem cell antigen 1-positive cardiac stromal cells. Journal of Pharmacology and Experimental Therapeutics. 2012; 341:764-774.
26. Moore M, Ryzhov S, Sawyer D B, Gartner C, Vary CPH. ALK1 Signaling in Human Cardiac Progenitor Cells Promotes a Pro-Angiogenic Secretome. J Cellular Signaling. 2024; In Press.
27. Sharma S, Mishra R, Bigham G E, Wehman B, Khan M M, Xu H, Saha P, Goo Y A, Datla S R, Chen L. A deep proteome analysis identifies the complete secretome as the functional unit of human cardiac progenitor cells. Circulation research. 2017; 120:816-834.
28. Arrell D K, Crespo-Diaz R J, Yamada S, Jeon R, Garmany A, Park S, Adolf J P, Livia C, Hillestad M L, Bartunek J. Secretome signature of cardiopoietic cells echoed in rescued infarcted heart proteome. Stem Cells Translational Medicine. 2021; 10:1320-1328.
29. Torin J L, Aguilar S, López J A, Torroja C, Quintana J A, Santiago C, Abad J L, Gomes-Alves P, Gonzalez A, Bernal J A. CXCL6 is an important paracrine factor in the pro-angiogenic human cardiac progenitor-like cell secretome. Scientific Reports. 2017; 7:12490.
30. Liu D, Cheng X, Wu H, Song H, Bu Y, Wang J, Zhang X, Yan C, Han Y. CREG1 attenuates doxorubicin-induced cardiotoxicity by inhibiting the ferroptosis of cardiomyocytes. Redox Biology. 2024; 75:103293.
31. Quast C, Alter C, Ding Z, Borg N, Schrader J. Adenosine formed by CD73 on T cells inhibits cardiac inflammation and fibrosis and preserves contractile function in transverse aortic constriction-induced heart failure. Circulation: Heart Failure. 2017; 10:e003346.
32. Li Q, Hou H, Li M, Yu X, Zuo H, Gao J, Zhang M, Li Z, Guo Z. CD73+ mesenchymal stem cells ameliorate myocardial infarction by promoting angiogenesis. Frontiers in cell and developmental biology. 2021; 9:637239.
33. Toomey B H, Mitrovic S A, Lindner-Liaw M, Vazquez R G L, Kacer D, Ryzhov S, Prudovsky I, Lindner V. Activated CTHRC1 promotes glycolysis in endothelial cells: Implications for metabolism and angiogenesis. Vascular pharmacology. 2023; 153:107246.
34. Simpson D L, Mishra R, Sharma S, Goh S K, Deshmukh S, Kaushal S. A strong regenerative ability of cardiac stem cells derived from neonatal hearts. Circulation. 2012; 126:S46-S53.
35. Grigorian-Shamagian L, Liu W, Fereydooni S, Middleton R C, Valle J, Cho J H, Marbin E. Cardiac and systemic rejuvenation after cardiosphere-derived cell therapy in senescent rats. European heart journal. 2017; 38:2957-2967.
36. Shen D, Cheng K, Marbin E. Dose-dependent functional benefit of human cardiosphere transplantation in mice with acute myocardial infarction. Journal of cellular and molecular medicine. 2012; 16:2112-2116.
37. Tang Y L, Tang Y, Zhang Y C, Qian K, Shen L, Phillips M I. Improved graft mesenchymal stem cell survival in ischemic heart with a hypoxia-regulated heme oxygenase-1 vector. Journal of the American College of Cardiology. 2005; 46:1339-1350.
38. Hong K U, Guo Y, Li Q-H, Cao P, Al-Magtari T, Vajravelu B N, Du J, Book M J, Zhu X, Nong Y. c-kit+ Cardiac stem cells alleviate post-myocardial infarction left ventricular dysfunction despite poor engraftment and negligible retention in the recipient heart. PloS one. 2014; 9:e96725.
39. Cheng K, Malliaras K, Smith R R, Shen D, Sun B, Blusztajn A, Xie Y, Ibrahim A, Aminzadeh M A, Liu W. Human cardiosphere-derived cells from advanced heart failure patients exhibit augmented functional potency in myocardial repair. JACC: Heart Failure. 2014; 2:49-61.
40. Ostovaneh M R, Makkar R R, Ambale-Venkatesh B, Ascheim D, Chakravarty T, Henry T D, Kowalchuk G, Aguirre F V, Kereiakes D J, Povsic T J. Effect of cardiosphere-derived cells on segmental myocardial function after myocardial infarction: ALLSTAR randomised clinical trial. Open Heart. 2021; 8:e001614.
41. Makkar R R, Smith R R, Cheng K, Malliaras K, Thomson L E, Berman D, Czer L S, Marbin L, Mendizabal A, Johnston P V. Intracoronary cardiosphere-derived cells for heart regeneration after myocardial infarction (CADUCEUS): a prospective, randomised phase 1 trial. The Lancet. 2012; 379:895-904.
42. McDonald C M, Marbin E, Hendrix S, Hogan N, Smith R R, Eagle M, Finkel R S, Tian C, Janas J, Harmelink M M. Repeated intravenous cardiosphere-derived cell therapy in late-stage Duchenne muscular dystrophy (HOPE-2): a multicentre, randomised, double-blind, placebo-controlled, phase 2 trial. The Lancet. 2022; 399:1049-1058.
43. Davis D R, Zhang Y, Smith R R, Cheng K, Terrovitis J, Malliaras K, Li T-S, White A, Makkar R, Marbin E. Validation of the cardiosphere method to culture cardiac progenitor cells from myocardial tissue. PloS one. 2009; 4:e7195.
44. Liu G, An L, Zhang H, Du P, Sheng Y. Activation of CXCL6/CXCR1/2 axis promotes the growth and metastasis of osteosarcoma cells in vitro and in vivo. Frontiers in pharmacology. 2019; 10:307.
45. Fan X, Patera A C, Pong-Kennedy A, Deno G, Gonsiorek W, Manfra D J, Vassileva G, Zeng M, Jackson C, Sullivan L. Murine CXCR1 is a functional receptor for GCP-2/CXCL6 and interleukin-8/CXCL8. Journal of Biological Chemistry. 2007; 282:11658-11666.
46. Dhayni K, Zibara K, Issa H, Kamel S, Bennis Y. Targeting CXCR1 and CXCR2 receptors in cardiovascular diseases. Pharmacology & Therapeutics. 2022; 237:108257.
47. Tan K, Zhu H, Zhang J, Ouyang W, Tang J, Zhang Y, Qiu L, Liu X, Ding Z, Deng X. CD73 expression on mesenchymal stem cells dictates the reparative properties via its anti-inflammatory activity. Stem cells international. 2019; 2019.
48. Ryzhov S, May T, Dziodzio J, Emery I F, Lucas F, Leclerc A, McCrum B, Lord C, Eldridge A, Robich M P. Number of Circulating C D 73-Expressing Lymphocytes Correlates With Survival After Cardiac Arrest. Journal of the American Heart Association. 2019; 8:e010874.
49. Monguió-Tortajada M, Roura S, Gilvez-Montón C, Franquesa M, Bayes-Genis A, Borris F E. Mesenchymal stem cells induce expression of CD73 in human monocytes in vitro and in a swine model of myocardial infarction in vivo. Frontiers in Immunology. 2017; 8:1577.
50. Ryzhov S, Biktasova A, Goldstein A E, Zhang Q, Biaggioni I, Dikov M M, Feoktistov I. Role of JunB in adenosine A2B receptor-mediated vascular endothelial growth factor production. Molecular pharmacology. 2014; 85:62-73.
51. Ryzhov S, Solenkova N V, Goldstein A E, Lamparter M, Fleenor T, Young P P, Greelish J P, Byrne J G, Vaughan D E, Biaggioni I. Adenosine receptor-mediated adhesion of endothelial progenitors to cardiac microvascular endothelial cells. Circulation research. 2008; 102:356-363.
52. Ruiz-Villalba A, Romero J P, Hernandez S C, Vilas-Zornoza A, Fortelny N, Castro-Labrador L, San Martin-Uriz P, Lorenzo-Vivas E, Garcia-Olloqui P, Palacio M, et al. Single-Cell RNA Sequencing Analysis Reveals a Crucial Role for CTHRC1 (Collagen Triple Helix Repeat Containing 1) Cardiac Fibroblasts After Myocardial Infarction. Circulation. 2020; 142:1831-1847. doi: 10.1161/CIRCULATIONAHA.119.044557
53. Wang D, Zhang Y, Ye T, Zhang R, Zhang L, Shi D, Li T, Xia G, Niu K, Zhao Z. Cthrc1 deficiency aggravates wound healing and promotes cardiac rupture after myocardial infarction via non-canonical WNT5A signaling pathway. International Journal of Biological Sciences. 2023; 19:1299.
54. Lu X, Wang Z, Ye D, Feng Y, Liu M, Xu Y, Wang M, Zhang J, Liu J, Zhao M. The role of CXC chemokines in cardiovascular diseases. Frontiers in Pharmacology. 2022; 12:765768.
55. Tecchio C, Cassatella M A. Neutrophil-derived cytokines involved in physiological and pathological angiogenesis. Angiogenesis, Lymphangiogenesis and Clinical Implications. 2014; 99:123-137.
56. Wysoczynski M, Solanki M, Borkowska S, Van Hoose P, Brittian K R, Prabhu S D, Ratajczak M Z, Rokosh G. Complement component 3 is necessary to preserve myocardium and myocardial function in chronic myocardial infarction. Stem Cells. 2014; 32:2502-2515.
57. Ratajczak M, Reca R, Wysoczynski M, Kucia M, Baran J, Allendorf D, Ratajczak J, Ross G. Transplantation studies in C3-deficient animals reveal a novel role of the third complement component (C3) in engraftment of bone marrow cells. Leukemia. 2004; 18:1482-1490.
58. Baehr A, Umansky K B, Bassat E, Jurisch V, Klett K, Bozoglu T, Hornaschewitz N, Solyanik O, Kain D, Ferraro B. Agrin promotes coordinated therapeutic processes leading to improved cardiac repair in pigs. Circulation. 2020; 142:868-881.
59. Bassat E, Mutlak Y E, Genzelinakh A, Shadrin I Y, Baruch Umansky K, Yifa O, Kain D, Rajchman D, Leach J, Riabov Bassat D. The extracellular matrix protein agrin promotes heart regeneration in mice. Nature. 2017; 547:179-184.
60. Wu Y, Chang T, Chen W, Wang X, Li J, Chen Y, Yu Y, Shen Z, Yu Q, Zhang Y.

Release of VEGF and BMP9 from injectable alginate based composite hydrogel for treatment of myocardial infarction. Bioactive materials. 2021; 6:520-528.
61. Bolli R, Hare J M, March K L, Pepine C J, Willerson J T, Perin E C, Yang P C, Henry T D, Traverse J H, Mitrani R D. Rationale and design of the CONCERT-H F trial (combination of mesenchymal and c-kit+ cardiac stem cells as regenerative therapy for heart failure). Circulation research. 2018; 122:1703-1715.
62. Kim J, Kim M, Jeong Y, Lee W-b, Park H, Kwon J-Y, Kim Y-M, Hwang D, Kwon Y-G. BMP9 induces cord blood-derived endothelial progenitor cell differentiation and ischemic neovascularization via ALK1. Arteriosclerosis, thrombosis, and vascular biology. 2015; 35:2020-2031.

1. A composition comprising a plurality of highly proliferative cells, wherein the plurality of highly proliferative cells express one or more of CXCL6, CTHRC1, and CD73, and wherein the highly proliferative cells are generated by clonal expansion of a cell.

2. The composition of claim 1, wherein the cell comprises a stem cell, a differentiated cell, or a heart cell.

3.-5. (canceled)

6. The composition of claim 1, wherein
the cell is derived from a biopsy.

7.-8. (canceled)

9. The composition of claim 1, further wherein:
(a) the plurality of highly proliferative cells express one or more of 1B46, CREG1, MRC1, TFPI2, CD200, PLXB2, or LRP1;
(b) the plurality of highly proliferative cells express one or more of ACTN4, APPL1, CAPZA1, CNN2, COL1A2, HIST2H3D, ISG15, MAOA, MTAP, MX1, PSMA2, PSMB4, PSMB7, PSMB9, PSME1, PYCARD, RIPK2, STAT1, STAT2, or YAP;
(c) the plurality of highly proliferative cells express one or more CXCL5, CXCL1, CXCL3, CXCL8, AGRIN, or CO3;
(d) the plurality of highly proliferative cells do not express c-kit and/or CD34; and/or
(e) the plurality of highly proliferative cells do not comprise CD45+ cells.

10.-13. (canceled)

14. The composition of claim 1, wherein:
(a) the highly proliferative cells are between about 25 μm to about 110 μm in size across at least one axis; and/or
(b) a concentration of a culture of the highly proliferative cells increases by at least 2-fold in about 15 hours to about 90 hours.

15.-17. (canceled)

18. The composition of claim 1, further comprising a medium suitable for maintaining the viability of the highly proliferative cells.

19. The composition of claim 1, wherein the composition is produced according to a method comprising:
providing a cell;
culturing the heart cell to form a colony;
selecting a colony, wherein the cells of the colony are between about 50 μm to about 110 μm in size, and wherein a concentration of a culture of the cells of the colony increases by at least 2-fold in about 15 hours to about 90 hours.

20.-23. (canceled)

24. The composition of claim 18, wherein the cell is cultured in a medium comprising fetal bovine serum (FBS) and an endothelial cell growth media.

25.-27. (canceled)

28. The composition of claim 19, wherein:
(a) CD45+ cells are depleted from the composition; and/or
(b) the composition comprises between about 0.25×10⁵ cells to about 5.0×10⁵ cells.

29.-31. (canceled)

32. A pharmaceutical composition comprising the composition of claim 1, and a pharmaceutically acceptable carrier.

33.-34. (canceled)

35. A method of producing the composition comprising a plurality of highly proliferative cells, the method comprising:
providing a cell;
culturing the cell to form a colony;
selecting a colony, wherein the cells of the colony are between about 50 μm to about 110 μm in size, and wherein a concentration of a culture of the cells of the colony increases by at least 2-fold in about 15 hours to about 90 hours; and
depleting CD45+ cells from the composition.

36. The method of claim 35, wherein the cell comprises a stem cell, a differentiated cell, or a heart cell.

37.-43. (canceled)

44. The method of claim 35, further wherein:
(a) the plurality of highly proliferative cells express one or more of 1B46, CREG1, MRC1, TFPI2, CD200, PLXB2, or LRP1;
(b) the plurality of highly proliferative cells express one or more of ACTN4, APPL1, CAPZA1, CNN2, COL1A2, HIST2H3D, ISG15, MAOA, MTAP, MX1, PSMA2, PSMB4, PSMB7, PSMB9, PSME1, PYCARD, RIPK2, STAT1, STAT2, or YAP;
(c) the plurality of highly proliferative cells express one or more CXCL5, CXCL1, CXCL3, CXCL8, AGRIN, or CO3;
(d) the plurality of highly proliferative cells do not express c-kit and/or CD34; and/or
(e) the cell is cultured in a medium comprising fetal bovine serum (FBS) and an endothelial cell growth media.

45.-48. (canceled)

49. A method of treating heart disease in a subject, the method comprising administering to the subject the composition of claim 1.

50. A method of improving cardiac function after myocardial infarction in a subject, the method comprising administering to the subject the composition of claim 1.

51. The method of claim 50, wherein cardiac function is measured by fractional shortening.

52.-53. (canceled)

54. A method of improving cardiac remodeling after myocardial infarction in a subject, the method comprising administering to the subject the composition of claim 1.

55. The method of claim 49, wherein:
(a) the composition is administered to the subject at least 10 minutes, at least 30 minutes, at least 1 hr, at least 2 hours, at least 3 hours, at least 5 hours, at least 12 hours, at least 24 hours, at least 48 hours, at least 72 hours after a myocardial infarction;
(b) about 1% to about 5% of the total number of cells in the composition are in the heart of the subject about 24 hours after administration; and/or
(c) the composition is injected into and/or adjacent to the subject's heart.

56.-57. (canceled)

58. (canceled)

59. (canceled)

60. The method of claim 49, wherein:
(a) at least 24 hours after administration of the composition CD45+ cells increase at the site of administration between about 1.5 fold to about 4 fold, about 1.5 fold to about 3.5 fold, about 1.5 fold to about 3 fold, about 1.5 fold to about 2.5 fold, about 1.5 fold to about 2 fold, about 2 fold to about 4 fold, about 2 fold to about 3.5 fold, about 2 fold to about 3 fold, about 2 fold to about 2.5 fold, about 2.5 fold to about 4 fold, about 2.5 fold to about 3.5 fold, about 2.5 fold to about 3 fold, about 3 fold to about 4 fold, about 3 fold to about 3.5 fold, or about 3.5 fold to about 4 fold compared to the level of CD45+ cells at the site of administration prior to administration; and/or
(b) at least 24 hours after administration of the composition granulocytic phagocytes increase at the site of administration by at least 1.5 fold, at least 2 fold, at least 2.5 fold, or at least 3 fold compared to the level of granulocytic phagocytes at the site of administration prior to administration.

61.-62. (canceled)

63. The method of claim 60, wherein the granulocytic phagocytes are CD11b+Ly6G+ cells.
